(19) United States
(12) Patent Application Publication
(10) Pub. No.: US 2023/0201358 A1
(43) Pub. Date: Jun. 29, 2023

(54) LINKER COMPOUNDS

(71) Applicant: MPEG LA, L.L.C., Chevy Chase, MD (US)

(72) Inventors: Jonathan Miles Brown, Silver Spring, MD (US); Kristin K.H. Neuman, Larchmont, NY (US)

(21) Appl. No.: 17/923,843

(22) PCT Filed: May 7, 2021
(86) PCT No.: PCT/US2021/031287

§ 371 Date: Nov. 7, 2022

Related U.S. Application Data
(60) Provisional application No. 63/022,313, filed on May 8, 2020.

(51) Int. Cl. A61K 47/54; A61K 47/55; A61K 47/68; C12N 15/113

(52) U.S. Cl. CPC A61K 47/549; A61K 47/55; A61K 47/6807; C12N 15/113; A61K 47/545; C12N 2310/14

(57) ABSTRACT

Linker compounds, methods of making them, and methods of using them as linking agents for oligonucleotides and other chemical and biological substances are described. Embodiments of linker compounds are configured or selected to exhibit higher stability to cleavage by serum nucleases relative to intracellular nucleases, enabling enhanced control of longevity and hence bioavailability to a target cell of the chemical and biological substances linked together by such linker compounds when administered to a subject.

### INCORPORATION BY REFERENCE TO PRIORITY APPLICATION

This application claims priority to U.S. Provisional Application Ser. No. 63/022,313, filed May 8, 2020, which is hereby incorporated herein by reference in its entirety.

### FIELD OF THE DISCLOSURE

The present disclosure relates to compounds, method of making the compounds, and related uses of the compounds as linking agents for oligonucleotides and other chemical and biological substances.

### BACKGROUND

Oligonucleotides are now a well-established class of therapeutics with multiple applications and ongoing clinical trials. However, many factors still limit the development and use of oligonucleotide therapeutics, for example, the delivery of the oligonucleotide to a target cell and the subsequent internalization of the oligonucleotide into the target cell in sufficient quantities to achieve a desired therapeutic effect.

To address this issue, oligonucleotides conjugated to ligands targeting specific cell surface receptors have been investigated. The use of one such ligand, N-acetylgalactosamine (GalNAc), has become a method of choice for oligonucleotide delivery to hepatocytes due to its highly specific and efficient binding to the asialoglycoprotein receptor, which is expressed in large numbers on the surface of these cells.

However even with the use of GalNAc-conjugated oligonucleotides, a high proportion of the compound is lost via excretion through the kidney. To counter this, multimers of oligonucleotides have been prepared wherein individual oligonucleotide subunits have been linked together via covalently bonded intermediates or “linkers”. These linkers have been introduced on the synthesizer or in aqueous solution after synthesis, deprotection and purification of the oligonucleotide.

A variety of linkers have been employed, including ones that are stable under in vivo conditions and others that are cleaved inside the target cell thereby liberating the individual oligonucleotide subunits. The most common type of cleavable linkers used have been short sequences of single-stranded unprotected nucleotides such as dTdTdTdT and dCdA, which are cleaved by intracellular nucleases, and disulfide-based linkers which are cleaved by the reductive environment inside the cell.

Another technique that has been successfully employed in the synthesis of multimeric oligonucleotides is asymmetric annealing whereby a single-stranded oligonucleotide bonded via a linker to another oligonucleotide is annealed to a complementary single-stranded oligonucleotide, optionally also bonded via a linker to another oligonucleotide, these steps being repeated until a multimer of the desired length is obtained.

Both homo- and hetero-multimers have been prepared via these methods and multimers in the 4-mer to 8-mer range exhibit notably enhanced serum half-lives and bioactivities.

However, these methods have limitations. Nuclease cleavable linkers can only be introduced via the synthesizer and generally only in 5′ to 3′ orientation, which limits the utility of the asymmetric annealing technique for the synthesis of multimeric oligonucleotides. Also, the presence of nucleic acid linking sequences immediately adjacent to the therapeutic oligonucleotide may impact the cleavability of the linker, the activity of the oligo, or both.

Disulfide linkages can be introduced both on the synthesizer and in aqueous solution after purification of the precursor. However, in the latter case, formation of disulfide bonds by reaction of thiols can lead to mixtures of products, especially with hetero systems. To avoid this problem, an alternative approach is to use an intermediate linking agent capable of reacting with thiol moieties which also contains a preformed internal disulfide bond. Such a linker is dithiobismaleimidoethane (DTME) which has an internal disulfide group and two terminal maleimide groups, each capable of reacting with a thiol group on another molecule.

DTME is normally used as a bivalent linker to link two identical thiolated entities to produce a homo-dimeric derivative. However, it has also been used to generate hetero-dimeric species via a monomeric intermediate wherein only one of the two maleimide moieties is allowed to react with a thiolated molecule. The resulting mono-DTME intermediate is then reacted with a second thiolated moiety to create a DTME linked hetero-dimer. This technique for the synthesis of a hetero-dimer is described in WO 2016/205410.

This methodology has been used to create multimeric oligonucleotides up to octamer in size in both homo- and hetero-multimeric forms.

However, certain aspects of disulfide bonds may be non-optimal for use in the synthesis of chemical compounds in general and in multimeric oligonucleotides in particular. For instance, it is not possible to maintain an internal disulfide group in a multimer while simultaneously reducing a terminal disulfide to a thiol for subsequent linking reactions.

Further, disulfide-linked molecules have been reported to dissociate and/or cross react with other thiolated species. In addition, long-term storage of disulfide-containing molecules can be problematic due to the potential for oxidation and subsequent cleavage of the disulfide bond.

There is therefore a need for additional methods and materials to act as linkers, which retain the advantages of cleavable linkers such as DTME without the perceived drawbacks of disulfide-containing molecules, in the assembly and synthesis of chemical compounds, including for example therapeutic agents and specifically including multimeric oligonucleotides.

### SUMMARY OF THE DISCLOSURE

The disclosure provides a linker compound comprising Structure 1:

X—R-□-R′—X′ (Structure 1)

wherein

X and X′ are each independently a functional group;

R and R′ are each independently a spacer group; and

□ is a covalent linker comprising at least one nucleotide.

In an embodiment of the linker compound, X and X′ are different functional groups; optionally, X and X′ are each independently a maleimide, azide, alkyne, activated carboxyl or amine.

In an embodiment of the linker compound, X and X′ are the same functional group; optionally, X and X′ are maleimide, azide, alkyne, activated carboxyl or amine.

In an embodiment of the linker compound, R and R′ are each independently an alkyl, alkyl ether, aryl, heteroaryl, heterocyclyl, alkyl-aryl, alkyl-heteroaryl, or alkyl-heterocyclyl.

In an embodiment, R and R′ are each independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl).

In an embodiment, R and R′ are each independently C₂-C₁₀ alkyl, C₂-C₁₀ alkyl ether, or C₆-C₁₀ aryl.

In an embodiment, R and R′ are each independently a C₂, C₃, C₄, C₅, or C₆ alkyl.

In an embodiment, R and R′ are C₆ alkyl.

In an embodiment, R and R′ are 1,4-phenylene.

In an embodiment of the linker compound, the covalent linker □ comprises at least two nucleotides; at least three nucleotides; or at least 4 nucleotides.

In an embodiment, the covalent linker □ comprises at least one inverted nucleotide.

In an embodiment, the covalent linker □ comprises at least two nucleotides that are the same.

In an embodiment, each nucleotide comprises uridine.

In an embodiment, each nucleotide comprises thymidine.

In an embodiment of the linker compound, the covalent linker □ comprises at least two nucleotides that are different from one another.

In an embodiment of the linker compound, the covalent linker □ comprises Structure 2:

[R″-(p)ₐ-N-(p)_{b}]_{c} (Structure 2)

wherein

c is an integer greater than or equal to 1; and

in each iteration of [R″-(p)ₐ-N-(p)_{b}]:
R″ is a spacer group or is absent;
each p is independently a derivative of phosphoric acid;
N is a nucleoside; and
a and b are each independently an integer greater than or equal to zero, with the proviso that a and b may not both be zero.

In an embodiment, c is an integer from 1 to 10.

In an embodiment, c is 2, 3, or 4.

In an embodiment, a and b are each independently 0, 1, 2 or 3, with the proviso that a and b may not both be 0.

In an embodiment, in each iteration of [R″-(p)ₐ-N-(p)_{b}], R″ is independently an alkyl, alkyl ether, aryl, heteroaryl, heterocyclyl, alkyl-aryl, alkyl-heteroaryl, alkyl-heterocyclyl, or is absent.

In an embodiment, in each iteration of [R″-(p)ₐ-N-(p)_{b}], R″ is independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl), or is absent.

In an embodiment, in each iteration of [R″-(p)ₐ-N-(p)_{b}], R″ is independently C₂-C₁₀ alkyl, C₂-C₁₀ alkyl ether, C₆-C₁₀ aryl, or is absent.

In an embodiment, in each iteration of [R″-(p)ₐ-N-(p)_{b}], R″ is independently a C₂, C₃, C₄, C₅ or C₂ alkyl, or is absent.

In an embodiment, in each iteration of [R″-(p)ₐ-N-(p)_{b}], R″ is independently C₆ alkyl or is absent.

In an embodiment, in each iteration of [R″-(p)ₐ-N-(p)_{b}], R″ is independently 1,4-phenylene, or is absent.

In an embodiment, in each iteration of [R″-(p)ₐ-N-(p)_{b}], each p is independently a phosphate, phosphorothioate, dithiophosphate, or phosphonate.

In an embodiment, at least one N is an inverted nucleoside.

In an embodiment, c is greater than or equal to 2 and at least two Ns are the same nucleoside.

In an embodiment, each N is uridine.

In an embodiment, each N is thymidine.

In an embodiment, c is greater than or equal to 2 and at least one N is different from another N.

In an embodiment of the linker compound, wherein Structure 2 is a compound according to Structure 3:

[pdTpdTpdTpdTp] (Structure 3),

wherein dT is thymidine.

In an embodiment of the linker compound, Structure 2 is a compound according to Structure 4:

[pUpUpUp] (Structure 4),

wherein U is uridine.

In an embodiment of the linker compound, the linker compound comprises Structure 5:

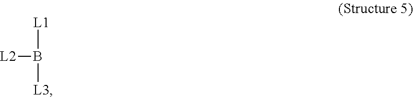

wherein:

B is a trivalent branch point; and

each of L1, L2 and L3 is, independently, Structure 6 or Structure 7:

*—R—[R″-(p)ₐ-N-(p)_{b}]_{c}-R′—X (Structure 6),

X—R—[R″-(p)ₐ-N-(p)_{b}]_{c}-R′—* (Structure 7)

wherein, in each of Structures 6 and 7:
* is the point of attachment to B;
each X is independently a functional group;
R and R′ are each independently a spacer group;
c is an integer greater than or equal to 1; and
in each iteration of [R″-(p)ₐ-N-(p)_{b}]:
R″ is a spacer group or is absent;
each p is independently a derivative of phosphoric acid;
N is a nucleoside;
a and b are each independently an integer greater than or equal to zero, with the proviso that a and b may not both be zero.

In an embodiment, one X in the linker compound is different from the other two Xs in the linker compound; optionally, each X is independently a maleimide, azide, alkyne, activated carboxyl or amine.

In an embodiment, each X in the linker compound is different from the other Xs in the linker compound; optionally, each X is independently a maleimide, azide, alkyne, activated carboxyl or amine.

In an embodiment, all of the Xs in the linker compound are the same; optionally, X is maleimide, azide, alkyne, activated carboxyl or amine.

In an embodiment, R and R′ are each independently an alkyl, alkyl ether, aryl, heteroaryl, heterocyclyl, alkyl-aryl, alkyl-heteroaryl, or alkyl-heterocyclyl.

In an embodiment, R and R′ are each independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl).

In an embodiment, R and R′ are each independently C₂-C₁₀ alkyl, C₂-C₁₀ alkyl ether, or C₆-C₁₀ aryl.

In an embodiment, R and R′ are each independently a C₂, C₃, C₄, C₅, or C₆ alkyl.

In an embodiment, R and R′ are C₆ alkyl.

In an embodiment, R and R′ are 1,4-phenylene.

In an embodiment, c is an integer from 1 to 10.

In an embodiment, c is 2, 3, or 4.

In an embodiment, a and b are each independently 0, 1, 2 or 3, with the proviso that a and b may not both be 0.

In an embodiment, in each iteration of [R″-(p)ₐ-N-(p)_{b}], R″ is independently an alkyl, alkyl ether, aryl, heteroaryl, heterocyclyl, alkyl-aryl, alkyl-heteroaryl, alkyl-heterocyclyl, or is absent.

In an embodiment, in each iteration of [R″-(p)ₐ-N-(p)_{b}], R″ is independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl), or is absent.

In an embodiment, in each iteration of [R″-(p)ₐ-N-(p)_{b}], R″ is independently C₂-C₁₀ alkyl, C₂-C₁₀ alkyl ether, C₆-C₁₀ aryl, or is absent.

In an embodiment, in each iteration of [R″-(p)ₐ-N-(p)_{b}], R′ is independently a C₂, C₃, C₄, C₅ or C₆ alkyl, or is absent.

In an embodiment, in each iteration of [R″-(p)ₐ-N-(p)_{b}], R′ is independently C₆ alkyl or is absent.

In an embodiment, each iteration of [R″-(p)ₐ-N-(p)_{b}], R″ is independently 1,4-phenylene, or is absent.

In an embodiment, in each iteration of [R″-(p)ₐ-N-(p)_{b}], each p is independently a phosphate, phosphorothioate, dithiophosphate, or phosphonate.

In an embodiment, at least one N is an inverted nucleoside.

In an embodiment, c is greater than or equal to 2 and at least two Ns are the same nucleoside.

In an embodiment, each N is uridine.

In an embodiment, each N is thymidine.

In an embodiment, c is greater than or equal to 2 and at least one N is different from another N.

In an embodiment, B is methanetriyl

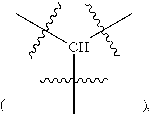 [SMILES: *C(*)*]

ethanetriyl

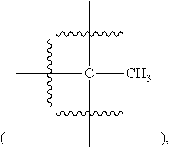 [SMILES: *C(*)(*)C]

propanetriyl

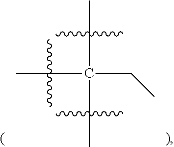 [SMILES: *C(*)(*)CC]

tris(hydroxymethyl)aminomethane, trisubstituted aryl, or substituted ammonia.

In an embodiment, B is methanetriyl

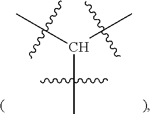 [SMILES: *C(*)*]

ethanetriyl

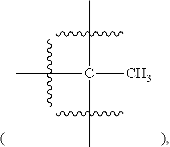 [SMILES: *C(*)(*)C]

propanetriyl

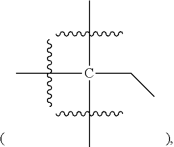 [SMILES: *C(*)(*)CC]

or tris(hydroxymethyl)aminomethane.

In an embodiment of any of the foregoing linker compounds, each nucleotide is independently a naturally-occurring nucleotide, optionally, a ribonucleotide or a deoxyribonucleotide; an artificial or non-natural nucleotide analog; or a chemically modified version of any of the foregoing.

In an embodiment of any of the foregoing linker compounds, each N is independently a naturally-occurring nucleoside, optionally, a ribonucleoside or a deoxyribonucleoside; an artificial or non-natural nucleoside analog, or a chemically modified version of any of the foregoing.

In an embodiment of any of the foregoing linker compounds, the compound is configured or selected to exhibit higher stability to cleavage by serum nucleases relative to intracellular nucleases.

In an embodiment of any of the foregoing linker compounds, the linker compound is at least about 75%, at least about 80%, at least about 85%, at least about 90%, at least about 95%, at least about 96%, at least about 97%, at least about 98%, at least about 99%, or about 100% pure.

In an embodiment of any of the foregoing linker compounds, the linker compound is about 85% to about 95% pure.

In an embodiment of any of the foregoing linker compounds, the linker compound is greater than or equal to 75% pure; greater than or equal to 85% pure; or greater than or equal to 95% pure.

The disclosure provides a multimeric oligonucleotide comprising subunits, wherein each of the subunits is independently a single-stranded or double-stranded oligonucleotide, and one or more of the subunits is joined to another subunit via covalent bonds formed by reaction with any of the foregoing linker compounds.

In an embodiment, each of the subunits is joined to an adjacent subunit via covalent bonds formed by reaction with any of the foregoing linker compounds.

In an embodiment, at least two subunits are substantially different.

In an embodiment, all the subunits are substantially the same.

In an embodiment, the multimeric oligonucleotide comprises two, three, four, five, or six subunits.

In an embodiment, each subunit is 15-30, 17-27, 19-26, or 20-25 nucleotides in length.

In an embodiment, one or more subunits are a double-stranded oligonucleotide.

In an embodiment, one or more subunits are a single-stranded oligonucleotide.

In an embodiment, one or more subunits are an antisense oligonucleotide.

In an embodiment, each subunit is, independently, an siRNA, a saRNA, or a miRNA.

In an embodiment, each subunit is a double-stranded siRNA.

In an embodiment, the multimeric oligonucleotide further comprises a targeting agent.

The disclosure provides a conjugate comprising a first bioactive compound joined to a second bioactive compound by reaction with any of the foregoing bivalent linker compounds.

In an embodiment, each of the first and second bioactive compounds is independently, a peptide, a protein, an oligonucleotide, an organometallic compound, or a small molecule drug.

In an embodiment, at least one of the bioactive compounds is an oligonucleotide.

In an embodiment, at least one of the bioactive compounds is an antibody or antibody fragment.

In an embodiment, the antibody is a monoclonal antibody.

In an embodiment, the first bioactive compound is a monoclonal antibody and the second bioactive compound is an oligonucleotide.

In an embodiment, the conjugate further comprises a targeting agent.

In an embodiment, the conjugate comprises two or more oligonucleotides linked together to form a multimeric oligonucleotide.

The disclosure provides a multi-conjugate comprising a first, second and third bioactive compound joined together by reaction with a trivalent linker compound comprising Structure 5.

In an embodiment, each of the first, second and third bioactive compounds is independently, a peptide, a protein, an oligonucleotide, an organometallic compound, or a small molecule drug.

In an embodiment, at least one of the bioactive compounds is an oligonucleotide.

In an embodiment, two of the bioactive compounds are each independently an oligonucleotide.

In an embodiment, at least one bioactive compound is an antibody or antibody fragment.

In an embodiment, the antibody is a monoclonal antibody.

In an embodiment, the first bioactive compound is a monoclonal antibody and the second and third bioactive compounds are each independently an oligonucleotide.

In an embodiment, the multi-conjugate further comprises a targeting agent.

In an embodiment, the multi-conjugate comprises two or more oligonucleotides linked together to form a multimeric oligonucleotide.

The disclosure provides a method for linking a first compound A to a second compound B comprising the steps of reacting any of the foregoing bivalent linker compounds with A and B, simultaneously or sequentially, under reaction conditions that promote the formation of a first covalent bond between A and the linker compound and a second covalent bond between B and the linker compound.

In an embodiment, A is different from B; and optionally, the terminal functional groups on the linker compound are different functional groups.

In an embodiment, A and B are the same; and optionally, the terminal functional groups on the linker compound are the same functional groups.

In an embodiment, A and B are each an oligonucleotide; optionally, siRNA.

In an embodiment, A is an oligonucleotide or a multimeric oligonucleotide and B is an antibody or antibody fragment.

In an embodiment, the oligonucleotide is siRNA.

The disclosure provides a method for linking compounds A, B and C together comprising the steps of reacting any of the foregoing trivalent linker compounds with each of A, B and C, simultaneously or sequentially, under reaction conditions that promote the formation of a covalent bond between the linker compound and each of A, B and C.

In an embodiment, at least one of A, B and C is different from the other two; and optionally, at least one functional group in the linker compound is a functional group that is different from the other two functional groups.

In an embodiment, one of A, B and C is an antibody and the other two are oligonucleotides; optionally, the antibody is a monoclonal antibody and the oligonucleotides are siRNA.

In an embodiment, all three compounds A, B and C are different; and optionally, each functional group in the linker compound is a different functional group.

In an embodiment, all three compounds A, B and C are the same; and optionally, each functional group in the linker compound is the same functional group.

The disclosure provides a method of treating a disease or condition in a subject comprising the step of administering to the subject an effective amount of a pharmaceutical composition comprising any of the foregoing multimeric oligonucleotides.

The disclosure provides a method of treating a disease or condition in a subject comprising the step of administering to the subject an effective amount of a pharmaceutical composition comprising any of the foregoing conjugates.

The disclosure provides a method of treating a disease or condition in a subject comprising the step of administering to the subject an effective amount of a pharmaceutical composition comprising any of the foregoing multi-conjugates.

The disclosure provides a composition comprising any of the foregoing multimeric oligonucleotides and a pharmaceutically acceptable excipient.

The disclosure provides a composition comprising any of the foregoing conjugates and a pharmaceutically acceptable excipient.

The disclosure provides a composition comprising any of the foregoing multi-conjugates and a pharmaceutically acceptable excipient.

The disclosure provides a composition comprising any of the foregoing multimeric oliognucleotides for use in the manufacture of a medicament.

The disclosure provides a composition comprising any of the foregoing conjugates for use in the manufacture of a medicament.

The disclosure provides a composition comprising any of the foregoing multi-conjugates for use in the manufacture of a medicament.

The disclosure provides a method of modulating the activity of a target gene in a cell, the method comprising contacting the cell with any of the foregoing multimeric oligonucleotides and maintaining the cell under conditions in which the multimeric oligonucleotide enters the cell and the activity of the target genes is modulated.

The disclosure provides a method of observing the activity of a bioactive compound in a cell, the method comprising contacting the cell with any of the foregoing conjugates and maintaining the cell under conditions in which the conjugate enters the cell and the activity of the bioactive compound is observed.

The disclosure provides a method of observing the activity of bioactive compound in a cell, the method comprising contacting the cell with any of the foregoing multi-conjugates and maintaining the cell under conditions in which the multi-conjugate enters the cell and the activity of the bioactive compound is observed.

### DETAILED DESCRIPTION

The disclosures of any patents, patent applications, and publications referred to herein are hereby incorporated by reference in their entireties into this application in order to more fully describe the state of the art known to those skilled herein as of the date of the disclosure described and claimed herein.

“Alkyl” refers to a straight or branched, saturated, aliphatic radical. The number of carbon atoms present in the alkyl group may be specified by number (e.g., C₃ alkyl contains three carbon atoms). The size range of an alkyl group can be specified by indicating a range of the numbers of carbon atoms (e.g., C₁-C₃ alkyl for a one to three carbon atom containing alkyl group). For example, C₁-C₆ alkyl includes, but is not limited to, methyl, ethyl, propyl, isopropyl, butyl, isobutyl, sec-butyl, tert-butyl, pentyl, isopentyl, hexyl, etc. Non-limiting examples of alkyl groups include methyl, ethyl, propyl, butyl, pentyl, 1-methylbutyl (i.e., 2-pentyl), 1-ethylpropyl (i.e., 3-pentyl), 3-methylpentyl, and the like. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6 carbons. The alkyl group is typically monovalent, but can be divalent, such as when the alkyl group links two moieties together, and it is understood that “alkyl” includes alkylene when two functionalities are appended.

“Alkyl ether” refers to a straight or branched chain saturated hydrocarbon containing 1-12 carbon atoms and 1-12 oxygen atoms in the chain. Examples of alkyl ethers include those represented by -((alkyl)-O—)— or —((CH₂)ₙ—O—)ₘ— where n is an integer in the range of 1 to 6 and m is an integer in the range of 1 to 12. A polyethylene glycol (PEG) group or linker is an example of an alkyl ether that may be represented by —((CH₂)₂—O—)ₘ—. An “alkoxy” is an example of an alkyl ether that contains a single oxygen atom attached to an end of the alkyl group e.g., —O-(alkyl). Examples of alkoxy groups include without limitation, methoxy, ethoxy, propoxy, butoxy, t-butoxy, or pentoxy groups.

“Aryl” refers to a monocyclic or fused bicyclic, tricyclic or greater, aromatic ring assembly containing 6 to 16 ring carbon atoms. Examples of aryl groups include, but are not limited to, phenyl, naphthyl, phenanthrenyl, naphthacenyl, fluorenyl, pyrenyl, and the like. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C2-C3-alkylene; all of which are optionally further substituted, for instance as hereinbefore defined; or 1- or 2-naphthyl; or 1- or 2-phenanthrenyl.

“Heteroaryl” refers to a monocyclic or fused bicyclic or tricyclic aromatic ring assembly containing 5 to 16 ring atoms, where from 1 to 4 of the ring atoms are each a heteroatom independently selected from N, O and S. Non-limiting examples of heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

“Heterocyclyl” refers to a ring system having from 3 ring members to about 20 ring members and from 1 to about 5 heteroatoms independently selected from N, O and S. For example, heterocyclyl includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

### Linker Compounds.

In addition to the linker compounds described in the Summary of the Disclosure, the present disclosure provides a linker compound of Structure 8:

X—R-pdTpdTpdTpdTp-R—X (Structure 8)

wherein each X is independently maleimide, azide, alkyne, activated carboxyl, or amine; each R is independently a C₂-C₆ alkyl; each p is independently a phosphate, phosphorothioate, dithiophosphate, or phosphonate; and dT is thymidine.

In an embodiment of the linker compound of Structure 8, each X is a maleimide.

In an embodiment of the linker compound of Structure 8, each X is azide.

In an embodiment of the linker compound of Structure 8, each X is alkyne.

In an embodiment of the linker compound of Structure 8, each X is activated carboxyl.

In an embodiment of the linker compound of Structure 8, each X is amine.

In an embodiment of the linker compound of Structure 8, one X is maleimide and the other X is, independently, azide, alkyne, activated carboxyl or amine.

In an embodiment of the linker compound of Structure 8, one X is azide and the other X is, independently, maleimide, alkyne, activated carboxyl or amine.

In an embodiment of the linker compound of Structure 8, one X is alkyne and the other X is, independently, maleimide, azide, activated carboxyl or amine.

In an embodiment of the linker compound of Structure 8, one X is activated carboxyl and the other X is, independently, maleimide, azide, alkyne, or amine.

In an embodiment of the linker compound of Structure 8, one X is amine and the other X is, independently, maleimide, azide, alkyne, or activated carboxy.

In an embodiment, the present disclosure provides a linker compound of Structure 9:

X—R-pUpUpUp-R—X (Structure 9)

wherein each X is independently maleimide, azide, alkyne, activated carboxyl, or amine; each R is independently a C₂-C₆ alkyl; each p is independently a phosphate, phosphorothioate, dithiophosphate, or phosphonate; and U is uridine.

In an embodiment of the linker compound of Structure 9, each X is maleimide.

In an embodiment of the linker compound of Structure 9, each X is azide.

In an embodiment of the linker compound of Structure 9, each X is alkyne.

In an embodiment of the linker compound of Structure 9, each X is activated carboxyl.

In an embodiment of the linker compound of Structure 9, each X is amine.

In an embodiment of the linker compound of Structure 9, one X is maleimide and the other X is, independently, azide, alkyne, activated carboxyl or amine.

In an embodiment of the linker compound of Structure 9, one X is azide and the other X is, independently, maleimide, alkyne, activated carboxyl or amine.

In an embodiment of the linker compound of Structure 9, one X is alkyne and the other X is, independently, a maleimide, azide, activated carboxyl or amine.

In an embodiment of the linker compound of Structure 9, one X is activated carboxyl and the other X is, independently, maleimide, azide, alkyne, or amine.

In an embodiment of the linker compound of Structure 9, one X is amine and the other X is, independently, maleimide, azide, alkyne, or activated carboxy.

In an embodiment, the present disclosure provides a linker compound of Structure 10:

Mal-R-pdTpdTpdTpdTp-R-Mal (Structure 10)

wherein Mal is a maleimide; each R is independently a C₂-C₆ alkyl; each p is independently phosphate, phosphorothioate, dithiophosphate, or phosphonate; and dT is thymidine.

In an embodiment, the present disclosure provides a linker compound of Structure 11:

Mal-R-pUpUpUp-R-Mal (Structure 11)

wherein Mal is a maleimide; each R is independently a C₂-C₆ alkyl; each p is independently phosphate, phosphorothioate, dithiophosphate, or phosphonate; and U is uridine.

In an embodiment, the present disclosure provides a linker compound of Structure 12:

X—R-pdTpdTpdTpdTp-R—X (Structure 12)

wherein each X is independently an alkyne; each R is independently a C₂-C₆ alkyl, each p is independently phosphate, phosphorothioate, dithiophosphate, or phosphonate; and dT is thymidine.

In an embodiment, the present disclosure provides a linker compound of Structure 13:

X—R-pUpUpUp-R—X (Structure 13)

wherein each X is independently an alkyne; each R is independently a C₂-C₆ alkyl, each p is independently phosphate, phosphorothioate, dithiophosphate, or phosphonate; and U is uridine.

In an embodiment, the present disclosure provides a linker compound of Structure 14:

X—R-pdTpdTpdTpdTp-R—X (Structure 14)

wherein each X is azide; each R is independently a C₂-C₆ alkyl; each p is independently phosphate, phosphorothioate, dithiophosphate, or phosphonate; and dT is thymidine.

In an embodiment, the present disclosure provides a linker compound of Structure 15:

X—R-pUpUpUp-R—X (Structure 15)

wherein each X is azide; each R is independently a C₂-C₆ alkyl; each p is independently phosphate, phosphorothioate, dithiophosphate, or phosphonate; and U is uridine.

In an embodiment, the present disclosure provides a linker compound of Structure 16:

X—R-pdTpdTpdTpdTp-R—X (Structure 16)

wherein each X is independently an activated carboxyl; each R is independently a C₂-C₆ alkyl; each p is independently phosphate, phosphorothioate, dithiophosphate, or phosphonate; and dT is thymidine.

In an embodiment, the present disclosure provides a linker compound of Structure 17:

X—R-pUpUpUp-R—X (Structure 17)

wherein each X is independently an activated carboxyl; each R is independently a C₂-C₆ alkyl; each p is independently phosphate, phosphorothioate, dithiophosphate, or phosphonate; and U is uridine.

In an embodiment, the present disclosure provides a linker compound of Structure 18:

X—R-pdTpdTpdTpdTp-R—X (Structure 18)

wherein each X is independently an amine; each R is independently a C₂-C₆ alkyl; each p is independently phosphate, phosphorothioate, dithiophosphate, or phosphonate; and dT is thymidine.

In an embodiment, the present disclosure provides a linker compound of Structure 19:

X—R-pUpUpUp-R—X (Structure 19)

wherein X is independently an amine; each R is independently a C₂-C₆ alkyl; each p is independently phosphate, phosphorothioate, dithiophosphate, or phosphonate; and U is uridine.

In an embodiment, the present disclosure provides a linker compound of Structure 20:

Mal-R-pdTpdTpdTpdTp-R—X (Structure 20)

wherein Mal is a maleimide; X is azide, alkyne, activated carboxyl or amine; each R is independently a C₂-C₆ alkyl; each p is independently phosphate, phosphorothioate, dithiophosphate, or phosphonate; and dT is thymidine.

In an embodiment, the present disclosure provides a linker compound of Structure 21:

Mal-R-pUpUpUp-R—X (Structure 21)

wherein Mal is a maleimide; X is azide, alkyne, activated carboxyl or amine; each R is independently a C₂-C₆ alkyl; each p is independently phosphate, phosphorothioate, dithiophosphate, or phosphonate; and U is uridine.

In an embodiment of any of the foregoing linker compounds, N is independently a naturally-occurring nucleoside (for example a ribonucleoside or a deoxyribonucleoside), an artificial or non-natural nucleoside analog, or a chemically modified version of any of the foregoing.

In an embodiment of any of the foregoing linker compounds, the linker compound is configured or selected to exhibit higher stability to cleavage by serum nucleases relative to intracellular nucleases.

In an embodiment of any of the foregoing linker compounds, the linker compound is isolated or substantially pure. For example, the compound can be at least about 75, 80, 85, 90, 95, 96, 97, 98, 99, or 100% pure. In one embodiment, the compound is about 85 to about 95% pure.

### Tunable Linker Compounds.

The present disclosure relates to embodiments of linker compounds that are configured or selected to exhibit higher stability to cleavage by serum nucleases relative to intracellular nucleases. This feature enables compounds linked together by such a linker compound to have enhanced longevity and hence bioavailability to a target cell when administered, yet still be readily released in active form after cell entry.

Nucleases—enzymes that cleave nucleic acids such as DNA and RNA—are ubiquitous in the human body where they form both a defense against infectious agents and also key parts of metabolic processes. Two main types of nuclease are known, exo-nucleases that degrade a nucleic acid from the termini and endo-nucleases that degrade a nucleic acid from the interior. Exo-nucleases are virtually the sole variety found in body fluids such as blood and serum, while both types are found inside the cells of the body. A key aspect of such embodiments of the disclosed linker compounds is resistance to exo-nucleases and simultaneous susceptibility to endo-nucleases.

In various embodiments, the linker compound is resistant to exo-nucleases as the linking functional groups at the termini are non-nucleic acid in nature and hence the whole linker is not susceptible to those enzymes. By contrast, the internal region of such a linker compound can contain one or more nucleic acid residues which are susceptible to endo-nucleases. This susceptibility can be increased or decreased according to preference by altering the number, type and position of the nucleosides, phosphoric acid derivatives and intervening spacer groups. Thus, by taking advantage of the higher lability of ribonucleotides to endo-nucleases, the linker may contain a UpUpUp sequence for rapid cleavage. Alternatively, the internal linker sequence may be dTp-alkyl-dTp for greater stability to endonuclease. In general a higher proportion of deoxy—rather than ribonucleotides, a greater proportion of spacer groups, and a higher proportion of phosphoric acid derivatives, as opposed to simple phosphates, results in a greater stability of the linker and a corresponding slower rate of cleavage by endo-nucleases. And vice versa.

In this way the biological characteristics of the linker compound can be “tuned” to the user's requirements.

### Conjugates and Multi-Conjugates Comprising the Linker Compounds.

The linker compound, as described above in all of its various embodiments, may be used in a linking or conjugation reaction to join various chemical or biological compounds, including, e.g., bioactive compounds. A bioactive compound is any molecule or agent that has a biological effect, in some cases a measurable biological effect. Bioactive compounds include, e.g., proteins, peptides, amino acids, nucleic acids, oligonucleotides, targeting agents, carbohydrates, polysaccharides, lipids, organic compounds, inorganic chemical compounds, organometallic compounds, small molecule drugs, detectable labels, and dervatives of any of the foregoing.

The term “detectable label” as used herein has its ordinary meaning as understood by those skilled in the art. It refers to a chemical group that is detectable by an imaging technique, such as fluorescence spectroscopy. For example, the detectable label may be a dye that comprises a fluorophore, which, after absorption of energy, emits radiation at a defined wavelength. Many suitable fluorescent labels or dyes are known. For example, Welch et al. (Chem. Eur. J. 5(3):951-960, 1999) discloses dansyl-functionalised fluorescent moieties and Zhu et al. (Cytometry 28:206-211, 1997) describes the use of the fluorescent labels Cy3 and Cy5. Other labels are described in Prober et al. (Science 238:336-341, 1987); Connell et al. (BioTechniques 5(4):342-384, 1987), Ansorge et al. (Nucl. Acids Res. 15(11):4593-4602, 1987) and Smith et al. (Nature 321:674, 1986). Examples of commercially available fluorescent labels include, but are not limited to, fluorescein, rhodamine (such as TMR, texas red or Rox), alexa, bodipy, acridine, coumarin, pyrene, benzanthracene and cyanine (such as Cy2 or Cy4). Other forms of detectable labels include microparticles, including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci USA 97(17):9461-9466, 2000), and tags detectable by mass spectrometry. The detectable label may be a multi-component label that is dependent on an interaction with another compound for detection, such as the biotin-streptavidin system.

Conjugates of bioactive compounds include, but are not limited to, antibody drug conjugates comprising an antibody or antibody fragment conjugated to a drug agent, including but not limited to a small molecule drug or an oligonucleotide therapeutic; other protein conjugates; and oligonucleotide conjugates. In an embodiment, the conjugates comprise oligonucleotides, polypeptides, or proteins involved in gene editing systems such as CRISPR/Cas, TALES, TALENS, and zinc finger nucleases (ZFNs).

In an embodiment, the conjugate comprises a first compound conjugated to a second compound via covalent bonds formed by reaction with a linker compound according to any of the various embodiments in the present disclosure, including but not limited to bivalent linker compounds according to any of Structures 1-4 and 6-21. In an embodiment, each of the first compound and the second compound is independently a protein, peptide, amino acid, nucleic acid, oligonucleotide, targeting agent, carbohydrate, polysaccharide, lipid, other organic compound, inorganic compound, organometallic compound, small molecule drug, or a derivative of any of the foregoing.

In a further embodiment, the conjugate comprises a multimeric oligonucleotide according to any of the embodiments described herein, or according to other types of multimeric oligonucleotides known in the art, including e.g., those made from different types of linkers and from different synthesis strategies (see, e.g., WO 2016/205410 A2; WO 2018/145086 A1; WO 2020/180897; WO 2021/026476; WO 2021/021959 A2 and WO 2021/026490, each of which is incorporated by reference herein in its entirety).

In an embodiment, the conjugate is an antibody or antibody fragment conjugated to an oligonucleotide or a multimeric oligonucleotide via covalent bonds formed by reaction with a linker compound according to any of the disclosed embodiments, including but not limited to the embodiments of Structures 1-4 and 6-21. In one such embodiment, the linker compound is a compound according to Structure 20:

Mal-R-pdTpdTpdTpdTp-R—X (Structure 20)

wherein Mal is a maleimide; X is azide, alkyne, activated carboxyl or amine; each R is independently a C₂-C₆ alkyl; each p is independently phosphate, phosphorothioate, dithiophosphate, or phosphonate; and dT is thymidine. In another embodiment, one terminal functional group in the linker compound (X) is maleimide and the other terminal functional group (X′) is cyclooctynyl. In an embodiment, the antibody is a monoclonal antibody; alternatively, the monoclonal antibody is a humanized monoclonal antibody. In an embodiment, the oligonucleotide or multimeric oligonucleotide comprises siRNA.

In other embodiments, the linker compound may be used in a series of linker or conjugation reactions to join multiple chemical or biological agents to form a “multi-conjugate.”

In an embodiment, the multi-conjugate comprises a first compound, a second compound, and a third compound conjugated together via covalent bonds formed by reaction with a multivalent linker compound according to any of the various embodiments in the present disclosure, including but not limited to a trivalent linker compound according to Structure 5. In an embodiment, each of the first, second and third compounds is independently a protein, peptide, amino acid, nucleic acid, oligonucleotide, targeting agent, carbohydrate, polysaccharide, lipid, other organic compound, inorganic compound, organometallic compound, small molecule drug, or a derivative of any of the foregoing.

In an embodiment of the multi-conjugate, each of the first, second and third compounds is independently an antibody, an antibody fragment, an oligonucleotide, or a multimeric oligonucleotide.

In an embodiment, the multiconjugate is a multimeric oligonucleotide comprised of two or more oligonucleotide “subunits” (each individually a “subunit”) wherein at least two subunits are linked together via covalent bonds formed by reaction with a linker compound according to any of the embodiments herein, whether bivalent as in Structures 1-4 and 6-21 or multivalent as in Structure 5. In an embodiment, the subunits may be multiple copies of the same subunit or differing subunits. In an embodiment, each of the subunits is independently a single-stranded or double-stranded oligonucleotide.

In an embodiment of the multimeric oligonucleotide, each of the subunits is joined to an adjacent subunit via covalent bonds formed by reaction with a linker compound according to any of the embodiments herein, whether bivalent as in Structures 1-4 and 6-21 or multivalent as in Structure 5.

In any of the foregoing multimeric oligonucleotides, at least two subunits are substantially different, alternatively, all of the subunits in the multimeric oligonucleotide are substantially different from one another.

In any of the foregoing multimeric oligonucleotides, at least two subunits are the same, alternatively, all of the subunits in the multimeric oligonucleotide are the same.

In an embodiment, the multimeric oligonucleotide comprises two, three, four, five, or six subunits.

In an embodiment of the multimeric oligonucleotide, each subunit is 15-30, 17-27, 19-26, or 20-25 nucleotides in length.

In an embodiment of the multimeric oligonucleotide, one or more subunits are a double-stranded RNA; alternatively, one or more subunits are a single-stranded RNA.

In an embodiment of the multimeric oligonucleotide, one or more subunits comprises DNA in single-stranded or double-stranded form.

In any of the foregoing multimeric oligonucleotides, one or more of the subunits are a single-stranded RNA or DNA; alternatively all of the subunits are a single-stranded RNA or DNA.

In an embodiment of the multimeric oligonucleotide, the subunits comprise a combination of single-stranded and double-stranded oligonucleotides.

In an embodiment of the multimeric oligonucleotide, each subunit is an siRNA, a saRNA, or a miRNA.

In an embodiment of the multimeric oligonucleotide, each subunit is a double-stranded siRNA.

In an embodiment, the multimeric oligonucleotide comprises two subunits of siRNA and the linker compound of Structure 8.

In any of the foregoing multimeric oligonucleotides, one or more of the subunits are an RNA or a DNA comprising a self-hybridizing, double-stranded segment, e.g., but not limited to an aptamer.

The conjugates, multiconjugates, and multimeric oligonucleotides may comprise all known types of nucleic acids, double-stranded and single-stranded, including for example, small interfering RNAs (siRNAs), small activating RNAs (saRNAs), microRNAs (miRNAs), antagomirs, CRISPR RNAs, long noncoding RNAs, piwi-interacting RNA, messenger RNA (mRNA), short hairpin RNA (shRNA), aptamers, ribozymes, and antisense oligonucleotides (for example, gapmers).

In an embodiment of any of the foregoing conjugates and multi-conjugates in which a linker compound comprising a terminal maleimide group

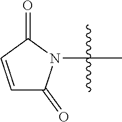 [SMILES: *N1C(=O)C=CC1=O]

is used in the formation of the conjugate or multi-conjugate, the maleimide group, upon reaction with a functionalized compound in the linking reaction, will form a closed-ring or an open-ring structure as follows;

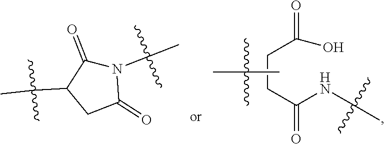

the latter structure being a composite structure representing the two possible open-ring positional isomers, which are derivatives of succinamic acid.

In an embodiment of any of the foregoing conjugate and multi-conjugate compounds, the compound is isolated or substantially pure. For example, the compound can be at least about 75, 80, 85, 90, 95, 96, 97, 98, 99, or 100% pure. In one embodiment, the compound is about 85 to about 95% pure.

### Methods of Making Conjugates and Multi-Conjugates.

The present disclosure relates to methods for linking a first compound to a second compound comprising the steps of reacting a linker compound with the first compound and the second compound, simultaneously or sequentially, under reaction conditions that promote the formation of a first covalent bond between the first compound and the linker compound and a second covalent bond between the second compound and the linker compound, wherein the linker compound comprises any of Structures 1-4 and 6-21.

In an embodiment, at least one of the first and second compounds is a bioactive compound; alternatively, both of the first and second compounds are a bioactive compound.

In an embodiment, the first compound is different from the second compound.

In an embodiment, the first compound is different from the second compound, and the linker compound comprises different terminal functional groups.

In an embodiment, the first compound and the second compound are the same.

In an embodiment, the first compound and the second compound are the same, and the linker compound comprises terminal functional groups that are the same.

In an embodiment, the first and second compound are an oligonucleotide. In an embodiment the first and second compounds are siRNA. In an embodiment, the first and second compounds are siRNA and the linker compound is Structure 8.

In an embodiment, the first compound is an oligonucleotide or a multimeric oligonucleotide and the second compound is an antibody. In an embodiment, the first compound is siRNA and the second compound is an antibody (e.g., a monoclonal antibody). In an embodiment, the first compound is siRNA, the second compound is an antibody (e.g., a monoclonal antibody) and the linker compound comprises Structure 18. In an embodiment, In an embodiment, the first compound is siRNA, the second compound is an antibody (e.g., a monoclonal antibody), and the linker compound comprises maleimide as one terminal functional group and cyclooctynyl as the other functional group.

The present disclosure relates to methods for linking three compounds together comprising the steps of reacting a trivalent linker compound with each of the three compounds, simultaneously or sequentially, under reaction conditions that promote the formation of a covalent bond between the trivalent linker compound and each of the three compounds, wherein the trivalent linker compound comprises Structure 5.

In an embodiment, at least one of the three compounds is a bioactive compound.

In an embodiment, all of the three compounds are a bioactive compound.

In an embodiment, at least one of the three compounds is different from the other compounds. In an embodiment, the trivalent linker compound comprises Structure 5 wherein at least one X is a functional group that is different from the other two Xs and optionally, one of the compounds is an antibody and the other two compounds are oligonucleotides, optionally siRNAs.

In an embodiment, each of the three compounds is different from the others. In an embodiment, the trivalent linker compound comprises Structure 5 wherein each X is a different functional group as compared to the other Xs.

### Nucleic Acids and Modifications.

In various embodiments, the nucleic acids of the linker compound and/or or the conjugates, multi-conjugates, or multimeric oligonucleotides may be modified using various strategies known in the art to produce a variety of effects, including, e.g., improved potency and stability in vitro and in vivo. Among these strategies are: chemically modified, artificial, and rare nucleic acids, including but not limited to 2′-O-methyl-substituted RNA and 2′-fluoro-2′deoxy RNA; peptide nucleic acid (PNA); morpholinos; locked nucleic acid (LNA); unlocked nucleic acid (UNA); bridged nucleic acid (BNA); glycol nucleic acid (GNA); threose nucleic acid (TNA); ribothymine, pseudouridine, methyl-pseudouridine, and mono- and dimethylguanine; or more generally, nucleic acid analogs, e.g., bicyclic and tricyclic nucleoside analogs, which are structurally similar to naturally occurring RNA and DNA but have alterations in one or more of the phosphate backbone, sugar, or nucleobase portions of the naturally-occurring molecule. Analogue nucleobases confer, among other things, different base pairing and base stacking properties. Examples include universal bases, which can pair with all four canonical bases.

In other embodiments, the nucleic acids may be modified to include nucleobase (often referred to in the art simply as “base”) modifications or substitutions. Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Kornberg, A., DNA Replication, W. H. Freeman & Co., San Francisco, pp 75-77 (1980); Gebeyehu et al., Nucl. Acids Res, 15: 4513 (1997). A “universal” base known in the art, e.g., inosine, can also be included. 5-me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, pp 276-278 (1993) and are aspects of base substitutions. Modified nucleobases can include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Other modifications include phosphorus-containing linkages, which include, but are not limited to, phosphorothioates, enantiomerically enriched phosphorothioates, phosphorodithioates, phosphotriesters, aminoalkylphosphotriesters, methyl and other alkyl phosphonates comprising 3′alkylene phosphonates and enantiomerically enriched phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted adjacent nucleoside units that are linked 3′-5′ to 5′-3′ or 2′-5′ to 5′-2′.

In various embodiments, the linker compound, conjugates, multiconjugates or multimeric oligonucleotides may comprise one or more phosphorothioate groups. Oligonucleotides may comprise 1-3 phosphorothioate groups at the 5′ end, or 1-3 phosphorothioate groups at the 3′ end, or 1-3 phosphorothioate groups at the 5′ end and the 3′ end. In various embodiments, each oligonucleotide may comprise 0-15 total phosphorothioate groups. In certain embodiments, each oligonucleotide may comprise fewer than 10, fewer than 9, fewer than 8, fewer than 7, fewer than 6, fewer than 5, fewer than 4, or fewer than 3 total phosphorothioate groups.

Hydroxy group (—OH) at a terminus of the nucleic acid can be substituted with a functional group such as sulfhydryl group (—SH), carboxyl group (—COOH) or amine group (—NH2), a formyl group (—CHO), a carbonyl group (—CO—), an ether group (—O—), an ester group (—COO—), a nitro group (—NO₂), an azide group (—N₃), or a sulfonic acid group (—SO₃H), an alkyne (—C≡C—), or an alkene (—CH═CH—). The substitution can be performed at the 3′ end or the 5′ end.

### Antibodies.

The present disclosure relates to linker compounds capable of linking an antibody to a therapeutic agent, or to multiple copies of a therapeutic agent (whether the same agent or different agents). The antibody may be a monoclonal antibody, a humanized antibody, or a fragment thereof. The linker compound may be used to bind a cysteine residue on the antibody or fragment via a maleimide group, while linked to the therapeutic agent via a different, orthogonal reactive group.

Alternatively, the linker compound may be linked to an antibody containing an unnatural amino acid such as p-azidomethyl-L-phenylalanine or azido-lysine via a cyclo-octyne group while linked to a therapeutic agent such as a thiolated siRNA via a maleimide group.

In all such cases precise stoichiometric amounts of a therapeutic agent or agents may be introduced for each linked amino acid on the antibody by utilization of branched forms of linker compounds comprising any of Structures 1-4 and 6-21, including but not limited to a trivalent linker compound comprising Structure 5. It is also possible to have differing intra-cellular release rates of each of the therapeutic agents bound to such a linker compound by having differing components in the various arms of the linker leading to differing rates of cleavage by endo-nucleases. In all cases, such rates of release are independent of the rate of degradation of the antibody itself.

### Targeting Agents.

Drug delivery systems have been designed using targeting ligands or conjugate systems to facilitate delivery to specific cells or tissues. For example, oligonucleotides can be conjugated to cholesterols, sugars, peptides, and other nucleic acids (e.g., aptamers) to facilitate delivery into specific cell types. Oftentimes, such conjugate systems facilitate delivery into specific cell types by binding to specific cell-surface receptors.

The linker compounds of the present disclosure may be used to conjugate a cell-targeting or tissue-targeting ligand or other targeting moiety (hereinafter, “targeting agent”) to a payload, which is any substance intended for intracellular or tissue delivery. The targeting agent may be made accessible on the surface of a nanoparticle, exosome, microvesicle, viral vector, other vector, carrier material or other delivery system (“package”) containing a payload for the purpose of delivering the package to a specific target. Alternatively, the targeting agent may be conjugated directly to the payload for direct delivery to the target without the need for formulation into a package.

Targeting agents within the scope of the present disclosure include but are not limited to an antibody, antibody fragment, double-chain antibody fragment, or single-chain antibody fragment; other protein, for example, a glycoprotein (e.g., transferrin) and a growth factor; a peptide, cell-penetrating peptide, viral or bacterial epitope, endosomal escape peptide or other endosomal escape agent; a chemical derivative of a peptide, for example 2-[3-(1,3-dicarboxypropyl)-ureido]pentanedioic acid (DUPA); a natural or synthetic carbohydrate, for example, a monosaccharide (e.g., galactose, mannose, N-Acetylgalactosamine [“GalNAc” ]), polysaccharide, or a cluster such as lectin binding oligo saccharide, diantennary GalNAc, or triantennary GalNAc; a lipid, for example, a sterol (e.g., cholesterol), phospholipid (e.g., phospholipid ether, phosphatidylcholine, lecithin), a vitamin compound (e.g., tocopherol or folate); immunostimulant (e.g., a CpG oligonucleotide); an amino acid (e.g., arginine-glycine-aspartic acid (“RGD”), a nucleic acid (e.g., an aptamer); an element (e.g., gold); and synthetic molecules (e.g., anisamide and polyethylene glycol). In an embodiment, the targeting agent comprises an aptamer, GalNAc, folate, lipid, cholesterol, or transferrin.

### Drug Delivery Systems.

As will be understood by those skilled in the art, regardless of biological target or mechanism of action, therapeutic oligonucleotides must overcome a series of physiological hurdles to access the target cell in an organism (e.g., animal, such as a human, in need of therapy). For example, a therapeutic oligonucleotide generally must avoid clearance in the bloodstream, enter the target cell type, and then enter the cytoplasm, all without eliciting an undesirable immune response. This process is generally considered inefficient, for example, 95% or more of siRNA that enters the endosome in vivo may be degraded in lysosomes or pushed out of the cell without affecting any gene silencing.

To overcome these obstacles, scientists have designed numerous drug delivery vehicles. These vehicles have been used to deliver therapeutic RNAs in addition to small molecule drugs, protein drugs, and other therapeutic molecules. Drug delivery vehicles have been made from materials as diverse as sugars, lipids, lipid-like materials, proteins, polymers, peptides, metals, hydrogels, conjugates, and peptides. Many drug delivery vehicles incorporate aspects from combinations of these groups, for example, some drug delivery vehicles can combine sugars and lipids. In some other examples, drugs can be directly hidden in ‘cell like’ materials that are meant to mimic cells, while in other cases, drugs can be put into, or onto, cells themselves. Drug delivery vehicles can be designed to release drugs in response to stimuli such as pH change, biomolecule concentration, magnetic fields, and heat.

Much work has focused on delivering oligonucleotides such as siRNA to the liver. The dose required for effective siRNA delivery to hepatocytes in vivo has decreased by more than 10,000 fold in the last ten years—whereas delivery vehicles reported in 2006 could require more than 10 mg/kg siRNA to target protein production, with new delivery vehicles target protein production can now be reduced after a systemic injection of 0.001 mg/kg siRNA. The increase in oligonucleotide delivery efficiency can be attributed, at least in part, to developments in delivery vehicles.

Another important advance has been an increased understanding of the way helper components influence delivery. Helper components can include chemical structures added to the primary drug delivery system. Often, helper components can improve particle stability or delivery to a specific organ. For example, nanoparticles can be made of lipids, but the delivery mediated by these lipid nanoparticles can be affected by the presence of hydrophilic polymers and/or hydrophobic molecules. One important hydrophilic polymer that influences nanoparticle delivery is poly(ethylene glycol). Other hydrophilic polymers include non-ionic surfactants. Hydrophobic molecules that affect nanoparticle delivery include cholesterol, 1-2-Distearoyl-sn-glycero-3-phosphocholine (DSPC), 1-2-di-O-octadecenyl-3-trimethylammonium propane (DOTMA), 1,2-dioleoyl-3-trimethylammonium-propane (DOTAP), and others.

One skilled in the art will appreciate that known delivery vehicles and targeting ligands can generally be adapted for use according to the present disclosure.

Examples of delivery vehicles and targeting ligands, as well as their use, can be found in: Sahay, G., et al. Efficiency of siRNA delivery by lipid nanoparticles is limited by endocytic recycling. Nat Biotechnol, 31: 653-658 (2013); Wittrup, A., et al. Visualizing lipid-formulated siRNA release from endosomes and target gene knockdown. Nat Biotechnol (2015); Whitehead, K. A., Langer, R. & Anderson, D. G. Knocking down barriers: advances in siRNA delivery. Nature reviews. Drug Discovery, 8: 129-138 (2009); Kanasty, R., Dorkin, J. R., Vegas, A. & Anderson, D. Delivery materials for siRNA therapeutics. Nature Materials, 12: 967-977 (2013); Tibbitt, M. W., Dahlman, J. E. & Langer, R. Emerging Frontiers in Drug Delivery. J Am Chem Soc, 138: 704-717 (2016); Akinc, A., et al. Targeted delivery of RNAi therapeutics with endogenous and exogenous ligand-based mechanisms. Molecular therapy: the journal of the American Society of Gene Therapy 18, 1357-1364 (2010); Nair, J. K., et al. Multivalent N-acetylgalactosamine-conjugated siRNA localizes in hepatocytes and elicits robust RNAi-mediated gene silencing. J Am Chem Soc, 136: 16958-16961 (2014); Ostergaard, M. E., et al. Efficient Synthesis and Biological Evaluation of 5′-GalNAc Conjugated Antisense Oligonucleotides. Bioconjugate chemistry (2015); Sehgal, A., et al. An RNAi therapeutic targeting antithrombin to rebalance the coagulation system and promote hemostasis in hemophilia. Nature Medicine, 21: 492-497 (2015); Semple, S. C., et al. Rational design of cationic lipids for siRNA delivery. Nat Biotechnol, 28: 172-176 (2010); Maier, M. A., et al. Biodegradable lipids enabling rapidly eliminated lipid nanoparticles for systemic delivery of RNAi therapeutics. Molecular therapy: the journal of the American Society of Gene Therapy, 21: 1570-1578 (2013); Love, K. T., et al. Lipid-like materials for low-dose, in vivo gene silencing. Proc Nat Acad USA, 107: 1864-1869 (2010); Akinc, A., et al. A combinatorial library of lipid-like materials for delivery of RNAi therapeutics. Nat Biotechnol, 26: 561-569 (2008); Eguchi, A., et al. Efficient siRNA delivery into primary cells by a peptide transduction domain-dsRNA binding domain fusion protein. Nat Biotechnol, 27: 567-571 (2009); Zuckerman, J. E., et al. Correlating animal and human phase Ia/Ib clinical data with CALAA-01, a targeted, polymer-based nanoparticle containing siRNA. Proc Nat Acad USA, 111: 11449-11454 (2014); Zuckerman, J. E. & Davis, M. E. Clinical experiences with systemically administered siRNA-based therapeutics in cancer. Nature Reviews. Drug Discovery, 14: 843-856 (2015); Hao, J., et al. Rapid Synthesis of a Lipocationic Polyester Library via Ring-Opening Polymerization of Functional Valerolactones for Efficacious siRNA Delivery. J Am Chem Soc, 29: 9206-9209 (2015); Siegwart, D. J., et al. Combinatorial synthesis of chemically diverse core-shell nanoparticles for intracellular delivery. Proc Nat Acad USA, 108: 12996-13001 (2011); Dahlman, J. E., et al. In vivo endothelial siRNA delivery using polymeric nanoparticles with low molecular weight. Nat Nano 9, 648-655 (2014); Soppimath, K S., Aminabhavi, T. M., Kulkami, A. R. & Rudzinski, W. E. Biodegradable polymeric nanoparticles as drug delivery devices. Journal of controlled release: official journal of the Controlled Release Society 70, 1-20 (2001); Kim, H. J., et al. Precise engineering of siRNA delivery vehicles to tumors using polyion complexes and gold nanoparticles. ACS Nano, 8: 8979-8991 (2014); Krebs, M. D., Jeon, O. & Alsberg, E. Localized and sustained delivery of silencing RNA from macroscopic biopolymer hydrogels. J Am Chem Soc 131, 9204-9206 (2009); Zimmermann, T. S., et al. RNAi-mediated gene silencing in non-human primates. Nature, 441: 111-114 (2006); Dong, Y., et al. Lipopeptide nanoparticles for potent and selective siRNA delivery in rodents and nonhuman primates. Proc Nat Acad USA, 111: 3955-3960 (2014); Zhang, Y., et al. Lipid-modified aminoglycoside derivatives for in vivo siRNA delivery. Advanced Materials, 25: 4641-4645 (2013); Molinaro, R., et al. Biomimetic proteolipid vesicles for targeting inflamed tissues. Nat Mater (2016); Hu, C. M., et al. Nanoparticle biointerfacing by platelet membrane cloaking. Nature, 526: 118-121 (2015): Cheng, R., Meng, F., Deng, C., Klok, H.-A. & Zhong, Z. Dual and multi-stimuli responsive polymeric nanoparticles for programmed site-specific drug delivery. Biomaterials, 34: 3647-3657 (2013); Qiu, Y. & Park, K. Environment-sensitive hydrogels for drug delivery. Advanced Drug Delivery Reviews, 64, Supplement, 49-60 (2012); Mui, B. L., et al. Influence of Polyethylene Glycol Lipid Desorption Rates on Pharmacokinetics and Pharmacodynamics of siRNA Lipid Nanoparticles. Mol Ther Nucleic Acids 2, e139 (2013); Draz, M. S., et al. Nanoparticle-Mediated Systemic Delivery of siRNA for Treatment of Cancers and Viral Infections. Theranostics, 4: 872-892 (2014); Otsuka, H., Nagasaki, Y. & Kataoka, K. PEGylated nanoparticles for biological and pharmaceutical applications. Advanced Drug Delivery Reviews, 55: 403-419 (2003); Kauffman, K. J., et al. Optimization of Lipid Nanoparticle Formulations for mRNA Delivery in vivo with Fractional Factorial and Definitive Screening Designs. Nano Letters, 15: 7300-7306 (2015); Zhang, S., Zhao, B., Jiang, H., Wang, B. & Ma, B. Cationic lipids and polymers mediated vectors for delivery of siRNA. Journal of Controlled Release 123, 1-10 (2007); IlIum, L. & Davis, S. S. The organ uptake of intravenously administered colloidal particles can be altered using a non-ionic surfactant (Poloxamer 338). FEBS Letters, 167: 79-82 (1984); Felgner, P. L., et al. Improved Cationic Lipid Formulations for In vivo Gene Therapy. Annals of the New York Academy of Sciences, 772: 126-139 (1995); Meade, B. R. & Dowdy, S. F. Exogenous siRNA delivery using peptide transduction domains/cell penetrating peptides. Advanced Drug Delivery Reviews, 59: 134-140 (2007); Endoh, T. & Ohtsuki, T. Cellular siRNA delivery using cell-penetrating peptides modified for endosomal escape. Advanced Drug Delivery Reviews, 61: 704-709 (2009); and Lee, H., et al. Molecularly self-assembled nucleic acid nanoparticles for targeted in vivo siRNA delivery. Nat Nano, 7: 389-393 (2012).

### Pharmaceutical Compositions Comprising the Linker Compounds.

The present disclosure relates to pharmaceutical compositions comprising an active pharmaceutical agent. In an embodiment, the active pharmaceutical agent can be joined to another substance or compound by a covalent bond formed by reaction with a bivalent linker compound as described herein, including but not limited to any of Structures 1-4 and 6-21, or a multivalent linker as described herein including but not limited to Structure 5. The active pharmaceutical agent may be a protein, peptide, amino acid, nucleic acid, targeting ligand, carbohydrate, polysaccharide, lipid, organic compound, or inorganic compound.

As used herein, pharmaceutical compositions include compositions of matter, other than foods, that contain one or more active pharmaceutical agents that can be used to prevent, diagnose, alleviate, treat, or cure a disease. Similarly, the various compounds or compositions according to the disclosure should be understood as including embodiments for use as a medicament and/or for use in the manufacture of a medicament.

A pharmaceutical composition can include a composition comprising an active pharmaceutical agent joined by a covalent bond formed by reaction with a linker compound as described herein, including but not limited to a linker compound of any of Structures 1-21, and a pharmaceutically acceptable excipient. As used herein, an excipient can be a natural or synthetic substance formulated alongside the active ingredient. Excipients can be included for the purpose of long-term stabilization, increasing volume (e.g., bulking agents, fillers, or diluents), or to confer a therapeutic enhancement on the active ingredient in the final dosage form, such as facilitating drug absorption, reducing viscosity, or enhancing solubility. Excipients can also be useful manufacturing and distribution, for example, to aid in the handling of the active ingredient and/or to aid in vitro stability (e.g., by preventing denaturation or aggregation). As will be understood by those skilled in the art, appropriate excipient selection can depend upon various factors, including the route of administration, dosage form, and active ingredient(s).

The pharmaceutical composition can be delivered locally or systemically, and the administrative route for pharmaceutical compositions of the disclosure can vary according to application. Administration is not necessarily limited to any particular delivery system and may include, without limitation, parenteral (including subcutaneous, intravenous, intramedullary, intraarticular, intramuscular, intraperitoneal, intraparenchymal, intracerebroventricular, and intrathecal, cisternal and lombar), rectal, topical, transdermal, or oral. Administration to an individual may occur in a single dose or in repeat administrations, and in any of a variety of physiologically acceptable salt forms, and/or with an acceptable pharmaceutical carrier and/or additive or adjuvant as part of a pharmaceutical composition. Physiologically acceptable formulations and standard pharmaceutical formulation techniques, dosages, and excipients are well known to persons skilled in the art (see, e.g., Physicians' Desk Reference (PDR®) 2005, 59th ed., Medical Economics Company, 2004; and Remington: The Science and Practice of Pharmacy, eds. Gennado et al. 21th ed., Lippincott, Williams & Wilkins, 2005).

Pharmaceutical compositions can include an effective amount of a conjugate or multi-conjugate made using a linker compound as described herein. As used herein, effective amount can be a concentration or amount that results in achieving a particular purpose, or an amount adequate to cause a change, for example in comparison to a placebo. Where the effective amount is a therapeutically effective amount, it can be an amount adequate for therapeutic use, for example an amount sufficient to prevent, diagnose, alleviate, treat, or cure a disease or condition. An effective amount can be determined by methods known in the art. An effective amount can be determined empirically, for example by human clinical trials. Effective amounts can also be extrapolated from one animal (e.g., mouse, rat, monkey, pig, dog) for use in another animal (e.g., human), using conversion factors known in the art. See, e.g., Freireich et al., Cancer Chemother Reports 50(4):219-244 (1966).

### Methods of Using Products Comprising the Linker Compounds.

The present disclosure also relates to methods of using compounds containing the above-described linker compounds in various applications, including but not limited to delivery to cells in vitro or in vivo for the purpose of modulating gene expression, biological research, treating or preventing medical conditions, and/or to produce new or altered phenotypes.

The present disclosure relates to methods of treating a disease or condition in a subject comprising the step of administering to the subject an effective amount of a pharmaceutical composition comprising an active pharmaceutical agent joined by a covalent bond to a linker compound as described herein, including but not limited to linker compounds according to any of Structures 1-21.

The present disclosure relates to methods for modulating gene expression, for example to silence, inhibit, or activate gene expression in a subject comprising the steps of administering to the subject an effective amount of a pharmaceutical composition comprising an active pharmaceutical agent joined by a covalent bond to a linker compound as described herein, including but not limited to linker compounds according to any of Structures 2-21. In an embodiment of this method, the active pharmaceutical agent is siRNA, saRNA, miRNA, antagomir, CRISPR RNA, long noncoding RNA, piwi-interacting RNA, messenger RNA, short hairpin RNA, aptamer, ribozyme, or antisense oligonucleotide (for example, a gapmer). In another embodiment, the linker compound may be conjugated to a protein or protein fragment involved in modulating gene expression, for example any of the CRISPR-Cas protein effectors (e.g., Cas9), TALES, TALENS, zinc finger nucleases, or derivatives of any of the foregoing.

In embodiments of these methods, the linker compound is conjugated to one or more of a protein (including but not limited to an antibody, monoclonal antibody, humanized antibody or fragments of the foregoing), peptide, amino acid, nucleic acid (including but not limited to an siRNA, saRNA, miRNA, antagomir, CRISPR RNA, long noncoding RNA, piwi-interacting RNA, messenger RNA, short hairpin RNA, aptamer, ribozyme, antisense oligonucleotide), targeting agent, carbohydrate, polysaccharide, lipid, organic compound, inorganic compound, organometallic compound, small molecule drug, imaging agent, or a derivative of any of the foregoing.

As used herein, a “subject” includes, but is not limited to, mammals, such as primates, rodents, and agricultural animals. Primate subjects include, but are not limited to, a human, a chimpanzee, and a rhesus monkey. Rodent subject includes, but are not limited to, a mouse and a rat. Agricultural animal subjects include, but are not limited to, a cow, a sheep, a lamb, a chicken, and a pig.

The following Examples are illustrative and not restrictive. Many variations of the technology will become apparent to those of skill in the art upon review of this disclosure. The scope of the technology should, therefore, be determined not with reference to the Examples, but instead should be determined with reference to the appended claims along with their full scope of equivalents.

### EXAMPLES

Oligoribonucleotides were assembled on ABI 394 and 3900 synthesizers (Applied Biosystems) at the 10 μmol scale, or on an Oligopilot 10 synthesizer at 28 μmol scale, using phosphoramidite chemistry. Solid supports were polystyrene loaded with 2′-deoxythymidine (Glen Research, Sterling, Va., USA), or controlled pore glass (CPG, 520 \acute{Å}, with a loading of 75 μmol/g, obtained from Prime Synthesis, Aston, Pa., USA). Ancillary synthesis reagents, DNA-, 2′-O-Methyl RNA-, and 2′-deoxy-2′-fluoro-RNA phosphoramidites were obtained from SAFC Proligo (Hamburg, Germany). Specifically, 5′-O-(4,4′-dimethoxytrityl)-3′-O-(2-cyanoethyl-N,N-diwasopropyl) phosphoramidite monomers of 2′-O-methyl-uridine (2′-OMe-U), 4-N-acetyl-2′-O-methyl-cytidine (2′-OMe-CAc), 6-N-benzoyl-2′-O-methyl-adenosine (2′-OMe-Abz) and 2-N-wasobutyrlguanosine (2′-OMe-GiBu) were used to build the oligomer sequences. 2′-Fluoro modifications were introduced employing the corresponding phosphoramidites carrying the same nucleobase protecting groups as the 2′-OMe RNA building blocks. Coupling time for all phosphoramidites (70 mM in Acetonitrile) was 3 min employing 5-Ethylthio-1H-tetrazole (ETT, 0.5 M in Acetonitrile) as activator. Phosphorothioate linkages were introduced using 50 mM 3-((Dimethylamino-methylidene)amino)-3H-1,2,4-dithiazole-3-thione (DDTT, AM Chemicals, Oceanside, Calif., USA) in a 1:1 (v/v) mixture of pyridine and Acetonitrile. Upon completion of the solid phase synthesis, including removal of the DMT group (“DMT off synthesis”), oligonucleotides were cleaved from the solid support and deprotected using a 1:1 mixture consisting of aqueous methylamine (41%) and concentrated aqueous ammonia (32%) for 3 hours at 25° C. according to published methods (Wincott, F. et al: Synthesis, deprotection, analysis and purification of RNA and ribozymes. Nucleic Acids Res, 23: 2677-2684 (1995).

Subsequently, crude oligomers were purified by anionic exchange HPLC using a column packed with Source Q15 (GE Healthcare) and an AKTA Explorer system (GE Healthcare). Buffer A was 10 mM sodium perchlorate, 20 mM Tris, 1 mM EDTA, pH 7.4 (Fluka, Buchs, Switzerland) in 20% aqueous Acetonitrile. Buffer B was the same as Buffer A with 500 mM sodium perchlorate. A gradient of 22% B to 42% B within 32 column volumes (CV) was employed. UV traces at 280 nm were recorded. Appropriate fractions were pooled and precipitated with 3M NaOAc, pH=5.2 and 70% Ethanol. Pellets were collected by centrifugation. Alternatively, desalting was carried out using Sephadex HiPrep columns (GE Healthcare) according to the manufacturer's recommendations.

Oligonucleotides were reconstituted in water and identity of the oligonucleotides was confirmed by electrospray ionization mass spectrometry (ESI-MS). Purity was assessed by analytical anion-exchange HPLC.

### Example 1: Preparation of Protected Tetra-Thymidine Triphosphate by Solid State Synthesis

A tetramer of thymidine with a dimethoxytrityl group at the 5′ end, a free hydroxyl at the 3′ end, and with each inter-nucleotide linkage protected by a 2-cyanoethyl group is prepared by solid state synthesis.

### Example 2: Preparation of Protected Tetra-Thymidine Triphosphate by Solution Chemistry

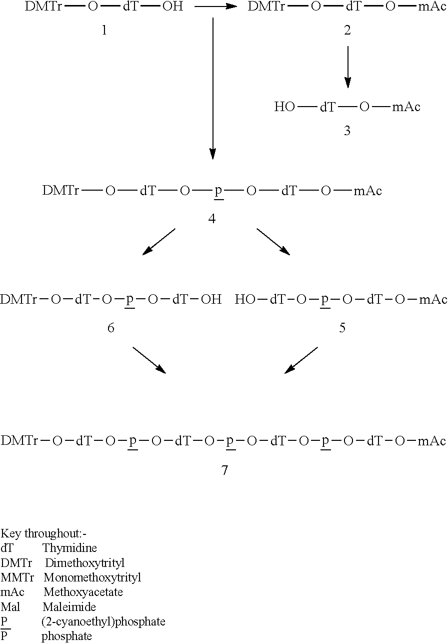

5′-O-dimethoxytritylthymidine (1) (Sigma Aldrich) is treated with methoxy-acetic anhydride in pyridine. After 1 hour the mixture is treated with saturated sodium bicarbonate and the mixture evaporated. The residue is partitioned between dichloromethane and sodium bicarbonate and the organic layer dried with magnesium sulfate and evaporated to dryness to yield 5′-O-dimethoxytrityl-3′-O′methoxyacetylthymidine (2).

This material is dissolved in tetrahydrofuran and treated with trichloroacetic acid to remove the dimethoxytrityl group. After neutralization with imidazole the mixture is evaporated to dryness and the residue washed with water and then dried under vacuum over phosphorus pentoxide to yield 3′-O-methoxyacetyl thymidine (3).

5′-O-dimethoxytritylthymidine 3′O-(2-cyanoethyl)-N,N-diisopropylphosphoramidite (Glen Research) is dissolved in acetonitrile and treated with tetrazole. After 5 minutes 3′-O-methoxyacetyl thymidine (2) in acetonitrile is added and the whole stirred for a further 15 minutes. Iodine in aqueous pyridine is then added and after a further 15 minutes aqueous sodium thiosulfate. The mixture is evaporated to dryness and the residue partitioned between dichloromethane and sodium bicarbonate and the organic layer dried with magnesium sulfate and evaporated to dryness. The residue is purified by short column chromatography on silica gel to yield the protected dithymidine phosphate 5′-O-dimethoxytritylthymidine 3′O-(2-cyanoethyl)phosphoro-5′-O-thymidine-3′-O-methoxyacetate (4).

This material is divided into two parts. The first part is treated with trichloroacetic acid to remove the dimethoxytrityl group. After neutralization with imidazole the mixture is evaporated to dryness and the residue washed with water and then dried under vacuum over phosphorus pentoxide to yield thymidine 3′-O-(2-cyanoethyl)phosphoro-5′-O-thymidine-3′-O-methoxyacetate (5).

The second part is treated with dilute methanolic ammonia for 5 minutes and then evaporated to dryness to yield the desired 5′-O-dimethoxytritylthymidine 3′O-(2-cyanoethyl)phosphoro-5′-O-thymidine (6). This material is dissolved in acetonitrile and treated with (2-cyanoethyl)-N,N-diisopropylchlorophosphoramidite and triethylamine. After 15 minutes the solution is treated with saturated sodium bicarbonate, evaporated to dryness and partitioned between saturated sodium bicarbonate and dichloromethane. The organic layer is dried and evaporated to dryness to yield the desired 5′-O-dimethoxytritylthymidine-3′O-(2-cyanoethyl)phosphoro-5′-O-thymidine-3′-O-(2-cyanoethyl)-N,N-diisopropylphosphoramidite.

This material is dissolved in acetonitrile and treated with tetrazole. After 5 minutes thymidine 3′-O-(2-cyanoethyl)phosphoro-5′-O-thymidine-3′-O-methoxyacetate (5) in acetonitrile is added and the whole stirred for a further 15 minutes. Iodine in aqueous pyridine is then added and after a further 15 minutes aqueous sodium thiosulfate. The mixture is evaporated to dryness and the residue partitioned between dichloromethane and sodium bicarbonate and the organic layer dried with magnesium sulfate and evaporated to dryness. The residue is purified by short column chromatography on silica gel to yield the required tetra-thymidine triphosphate with a dimethoxytrityl group at the 5′ end, a methoxyacetate at the 3′ end, and with each inter-nucleotide linkage protected by a 2-cyanoethyl group (7).

### Example 3: Preparation of a Homo-Bifunctional Linker Compound with Terminal Maleimide Moieties

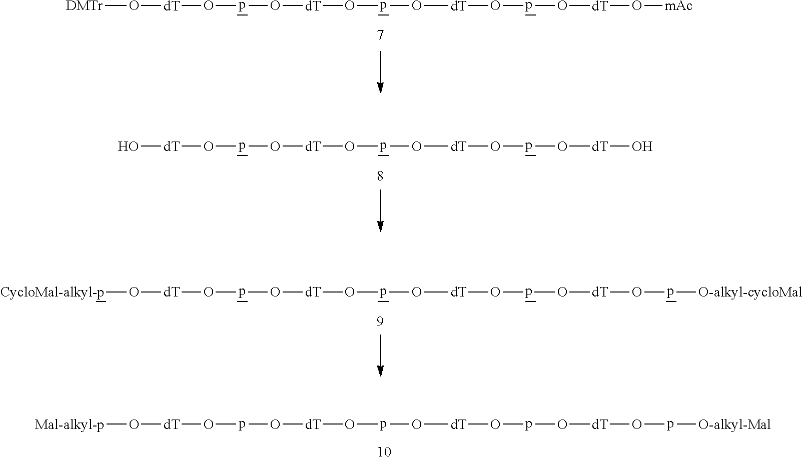

Tetra-thymidine triphosphate with 5′ and 3′-terminal maleimide moieties (10) is prepared by sequentially treating the fully protected material (7) prepared in Example 2 dissolved in dioxane with i) dilute methanolic ammonia and ii) trichloroacetic acid followed by neutralization with imidazole. After workup the product (8) is treated with two equivalents of 2-(1,7-Dimethyl-3,5-dioxo-10-oxa-4-azatricyclo[5.2.1.02,6]dec-8-en-4-yl)-ethyl-1-O-[(2-cyanoethyl)-(N,N-diisopropyl)]-phosphoramidite (Glen Research) and tetrazole, followed by iodine in aqueous pyridine to introduce the two terminal protected maleimide groups (9). Treatment of this material with strictly anhydrous tetramethylguanidine in dioxane removes the cyanoethyl protecting groups from the inter-nucleotide linkages. After purification the product is suspended in strictly anhydrous toluene and heated to 90 deg C. to unprotect the terminal maleimides and yield the desired homo-bifunctional linker compound (10).

### Example 4: Preparation of a Homo-Bifunctional Linker Compound with Terminal Cyclooctynyl Moieties

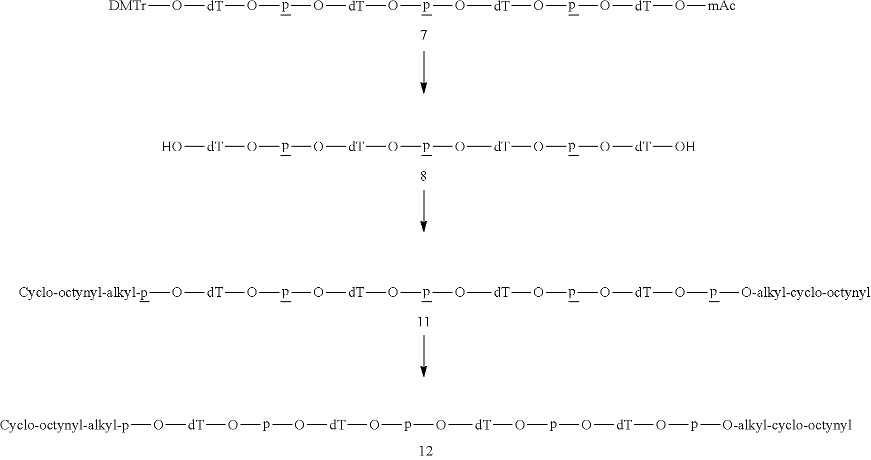

Tetra-thymidine triphosphate with 5′ and 3′-terminal cyclooctynyl moieties (12) is prepared by sequentially treating the fully protected material prepared in Example 2 (7) in dioxane with i) dilute methanolic ammonia and ii) trichloroacetic acid followed by neutralization with imidazole. After workup the product (8) is treated with two equivalents of 10-(6-oxo-6-(dibenzo[b,f]azacyclooct-4-yn-1-yl)-capramido-N-ethyl)-O-triethyleneglycol-1-[(2-cyanoethyl)-(N,N-diisopropyl)]-phosphoramidite (Glen Research) and tetrazole, followed by iodine in aqueous pyridine to introduce two terminal cyclooctynyl groups (11). Treatment of this material with strictly anhydrous tetramethylguanidine in dioxane removes the cyanoethyl protecting groups from the inter-nucleotide linkages to yield the desired homo-bifunctional linker compound (12) after purification.

### Example 5: Preparation of a Hetero-Bifunctional Linker Compound with 5′-Maleimide and a 3′-Cydooctyne Moieties

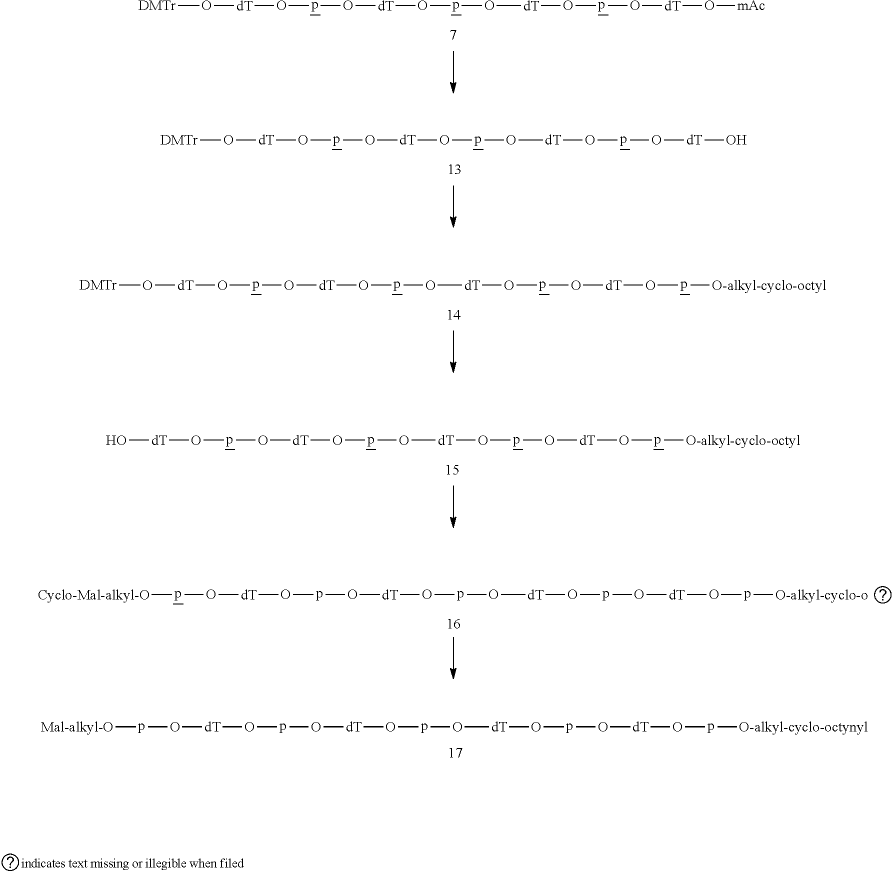

Tetra-thymidine triphosphate with 5′- and 3′-terminal maleimide and cyclooctyne moieties, respectively, (17) is prepared by treating the fully protected material (7) prepared in Example 2 dissolved in dioxane with dilute methanolic ammonia and evaporating to dryness. The product (13) is treated with 10-(6-oxo-6-(dibenzo[b,f]azacyclooct-4-yn-1-yl)-capramido-N-ethyl)-O-triethyleneglycol-1-[(2-cyanoethyl)-(N,N-diisopropyl)]-phosphoramidite (Glen Research) to add the cyclooctyne moiety to the 3′ end (14).

Removal of the 5′-dimethoxytrityl group is achieved by treatment of (14) in dichloromethane with trichloroacetic acid followed by imidazole to yield the 5′-unprotected tetramer (15). This material is treated with 2-(1,7-Dimethyl-3,5-dioxo-10-oxa-4-azatricyclo[5.2.1.02,6]dec-8-en-4-yl)-ethyl-1-O-[(2-cyanoethyl)-(N,N-diisopropyl)]-phosphoramidite (Glen Research) and tetrazole, followed by iodine in aqueous pyridine to introduce a protected maleimide group to the 5′-end (16). Treatment of this material with strictly anhydrous tetramethylguanidine in dioxane removes the cyanoethyl protecting groups from the inter-nucleotide linkages. After purification the product is suspended in strictly anhydrous toluene and heated to 90 deg C. to unprotect the terminal maleimide and yield the desired hetero-bifunctional linker compound (17).

### Example 6: Preparation of a Hetero-Bifunctional Linker Compound with 5′-Amino and 3′-Cyclooctyne Moieties

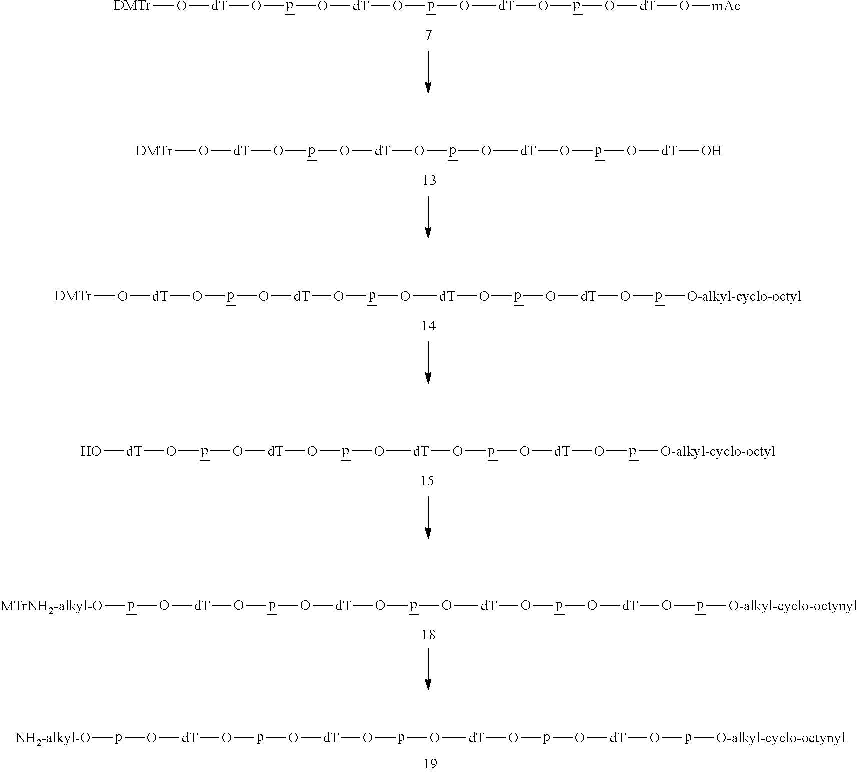

Tetra-thymidine triphosphate with 5′- and 3′-terminal amino and cyclooctyne moieties, respectively, (19) is prepared by treating the fully protected material (7) prepared in Example 2 dissolved in dioxane with dilute methanolic ammonia and evaporated to dryness. The product (13) is treated with 10-(6-oxo-6-(dibenzo[b,f]azacyclooct-4-yn-1-yl)-capramido-N-ethyl)-O-triethyleneglycol-1-[(2-cyanoethyl)-(N,N-diisopropyl)]-phosphoramidite (Glen Research) to add the cyclooctyne moiety to the 3′ end (14). Removal of the 5′-dimethoxytrityl group is achieved by treatment of (14) with trichloroacetic acid followed by imidazole to yield the 5′-unprotected tetramer (15). This material is treated with two equivalents of 6-(4-Monomethoxytritylamino)hexyl-(2-cyanoethyl)-(N,N-diisopropyl)-phosphoramidite (Glen Research) followed by iodine in aqueous pyridine to introduce a protected amino group to the 5′-end (18). Treatment of this material in dioxane with i) strictly anhydrous tetramethylguanidine and ii) trichloroacetic acid in dioxane removes the protecting groups from the inter-nucleotide linkages and amino function, respectively, to yield the desired hetero-bifunctional linker compound (19).

### Example 7: Conjugation of siRNA Via a Bis(Maleimide) Linker Compound

Tetra-thymidine triphosphate with 5′ and 3′-terminal maleimide groups (10) (prepared in Example 3) is dissolved in triethylammonium bicarbonate buffer (TEABc, 0.1M, pH 8.5, Sigma-Aldrich) and treated with two equivalents of an siRNA with a terminal 3′-thiol group in the same buffer. After 15 minutes the buffer is removed by evaporation to yield the required dimerized siRNA.

### Example 8: Conjugation of siRNA to an Antibody Via a Maleimide-Cyclooctynyl Linker Compound

Tetra-thymidine triphosphate with 5′- and 3′-terminal maleimide and cyclooctynyl groups, respectively, (17) is dissolved in triethylammonium bicarbonate buffer (TEABc, 0.1M, pH 8.5, Sigma-Aldrich) and treated with one equivalent of an siRNA with a terminal 3′-azide group in the same buffer. After 15 minutes a solution of an antibody with a free thiol group is added and the whole stirred at room temperature for an hour. The desired antibody-linker-siRNA conjugate is isolated by preparative chromatography.

1. A linker compound comprising Structure 1:
X—R-□-R′—X′ (Structure 1)
wherein
X and X′ are each independently a functional group;
R and R′ are each independently a spacer group; and
□ is a covalent linker comprising at least one nucleotide.

2. The linker compound of claim 1, wherein X and X′ are different functional groups.

3. The linker compound of claim 2, wherein X and X′ are each independently a maleimide, azide, alkyne, activated carboxyl or amine.

4. The linker compound of claim 1, wherein X and X′ are the same functional group.

5. The linker compound of claim 4, where X and X′ are maleimide, azide, alkyne, activated carboxyl or amine.

6. The linker compound of any of claims 1-5, wherein R and R′ are each independently an alkyl, alkyl ether, aryl, heteroaryl, heterocyclyl, alkyl-aryl, alkyl-heteroaryl, or alkyl-heterocyclyl.

7. The linker compound of claim 6, wherein R and R′ are each independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl).

8. The linker compound of claim 6, wherein R and R′ are each independently C₂-C₁₀ alkyl, C₂-C₁₀ alkyl ether, or C₆-C₁₀ aryl.

9. The linker compound of claim 6, wherein R and R′ are each independently a C₂, C₃, C₄, C₅, or C₆ alkyl.

10. The linker compound of claim 6, wherein R and R′ are C₆ alkyl.

11. The linker compound of claim 6, wherein R and R′ are 1,4-phenylene.

12. The linker compound of any of claims 1-11, wherein the covalent linker □ comprises at least two nucleotides; at least three nucleotides; or at least 4 nucleotides.

13. The linker compound of any of claims 1-12, wherein the covalent linker □ comprises at least one inverted nucleotide.

14. The linker compound of any of claims 1-12, wherein the covalent linker □ comprises at least two nucleotides that are the same.

15. The linker compound of claim 14, wherein each nucleotide comprises uridine.

16. The linker compound of claim 14, wherein each nucleotide comprises thymidine.

17. The linker compound of any of claims 1-13, wherein the covalent linker □ comprises at least two nucleotides that are different from one another.

18. The linker compound of any of claims 1-17, wherein the covalent linker Z comprises Structure 2:
[R″-(p)ₐ-N-(p)_{b}]_{c} (Structure 2)
wherein
c is an integer greater than or equal to 1; and
in each iteration of [R″-(p)ₐ-N-(p)_{b}]:
R″ is a spacer group or is absent;
each p is independently a derivative of phosphoric acid;
N is a nucleoside;
a and b are each independently an integer greater than or equal to zero, with the proviso that a and b may not both be zero; optionally, a and b are each independently 0, 1, 2 or 3, with the proviso that a and b may not both be 0.

19. The linker compound of claim 18, wherein c is an integer from 1 to 10.

20. The linker compound of claim 19, wherein c is 2, 3, or 4.

21. The linker compound of any of claims 18-20, wherein in each iteration of [R″-(p)ₐ-N-(p)_{b}], R″ is independently an alkyl, alkyl ether, aryl, heteroaryl, heterocyclyl, alkyl-aryl, alkyl-heteroaryl, alkyl-heterocyclyl, or is absent.

22. The linker compound of claim 21, wherein in each iteration of [R″-(p)ₐ-N-(p)_{b}], R″ is independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl), or is absent.

23. The linker compound of claim 21, wherein in each iteration of [R″-(p)ₐ-N-(p)_{b}], R″ is independently C₂-C₁₀ alkyl, C₂-C₁₀ alkyl ether, C₆-C₁₀ aryl, or is absent.

24. The linker compound of claim 21, wherein in each iteration of [R″-(p)ₐ-N-(p)_{b}], R″ is independently a C₂, C₃, C₄, C₅ or C₆ alkyl, or is absent.

25. The linker compound of claim 21, wherein in each iteration of [R″-(p)ₐ-N-(p)_{b}], R″ is independently C₆ alkyl or is absent.

26. The linker compound of claim 21, wherein in each iteration of [R″-(p)ₐ-N-(p)_{b}], R″ is independently 1,4-phenylene, or is absent.

27. The linker compound of any of claims 18-26, wherein in each iteration of [R″-(p)ₐ-N-(p)_{b}], each p is independently a phosphate, phosphorothioate, dithiophosphate, or phosphonate.

28. The linker compound of any of claims 18-27, wherein at least one N is an inverted nucleoside.

29. The linker compound of any of claims 18-27, wherein c is greater than or equal to 2 and at least two Ns are the same nucleoside.

30. The linker compound of claim 29, wherein each N is uridine.

31. The linker compound of claim 29, wherein each N is thymidine.

32. The linker compound of any of claims 18-28, wherein c is greater than or equal to 2 and at least one N is different from another N.

33. The linker compound claim 29, wherein Structure 2 is a compound according to Structure 3:
[pdTpdTpdTpdTp] (Structure 3),
wherein dT is thymidine.

34. The linker compound of claim 29, wherein Structure 2 is a compound according to Structure 4:
[pUpUpUp] (Structure 4),
wherein U is uridine.

35. A linker compound of Structure 5: 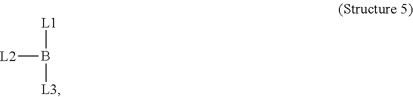
wherein:
B is a trivalent branch point;
each of L1, L2 and L3 is, independently, Structure 6 or Structure 7:
*—R—[R″-(p)ₐ-N-(p)_{b}]_{c}—R′—X (Structure 6),
X—R—[R″-(p)ₐ-N-(p)_{b}]_{c}-R′—* (Structure 7)
wherein, in each of Structures 6 and 7:
* is the point of attachment to B;
each X is independently a functional group;
R and R′ are each independently a spacer group;
c is an integer greater than or equal to 1; and
in each iteration of [R″-(p)ₐ-N-(p)_{b}]:
R″ is a spacer group or is absent;
each p is independently a derivative of phosphoric acid;
N is a nucleoside;
a and b are each independently an integer greater than or equal to zero, with the proviso that a and b may not both be zero; optionally, a and b are each independently 0, 1, 2 or 3, with the proviso that a and b may not both be 0.

36. The linker compound of claim 35, wherein one X in the linker compound is different from the other two Xs in the linker compound.

37. The linker compound of claim 35, wherein each X in the linker compound is different from the other Xs in the linker compound.

38. The linker compound of claim 36 or 37, wherein each X is independently a maleimide, azide, alkyne, activated carboxyl or amine.

39. The linker compound of claim 35, wherein all of the Xs in the linker compound are the same.

40. The linker compound of claim 39, where X is maleimide, azide, alkyne, activated carboxyl or amine.

41. The linker compound of any of claims 35-40, wherein R and R′ are each independently an alkyl, alkyl ether, aryl, heteroaryl, heterocyclyl, alkyl-aryl, alkyl-heteroaryl, or alkyl-heterocyclyl.

42. The linker compound of claim 41, wherein R and R′ are each independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl).

43. The linker compound of claim 41, wherein R and R′ are each independently C₂-C₁₀ alkyl, C₂-C₁₀ alkyl ether, or C₆-C₁₀ aryl.

44. The linker compound of claim 41, wherein R and R′ are each independently a C₂, C₃, C₄, C₅, or C₆ alkyl.

45. The linker compound of claim 41, wherein R and R′ are C₆ alkyl.

46. The linker compound of claim 41, wherein R and R′ are 1,4-phenylene.

47. The linker compound of any of claims 35-46, wherein c is an integer between 1 and 10.

48. The linker compound of claim 47, wherein c is 2, 3, or 4.

49. The linker compound of any of claims 35-48, wherein in each iteration of [R″-(p)ₐ-N-(p)_{b}], R″ is independently an alkyl, alkyl ether, aryl, heteroaryl, heterocyclyl, alkyl-aryl, alkyl-heteroaryl, alkyl-heterocyclyl, or is absent.

50. The linker compound of claim 49, wherein in each iteration of [R″-(p)ₐ-N-(p)_{b}], R′ is independently a C₁₋₁₀ alkyl, C₁₋₁₀ alkyl ether, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl), or is absent.

51. The linker compound of claim 49, wherein in each iteration of [R″-(p)ₐ-N-(p)_{b}], R″ is independently C₂-C₁₀ alkyl, C₂-C₁₀ alkyl ether, C₆-C₁₀ aryl, or is absent.

52. The linker compound of claim 49, wherein in each iteration of [R″-(p)ₐ-N-(p)_{b}], R″ is independently a C₂, C₃, C₄, C₅ or C₆ alkyl, or is absent.

53. The linker compound of claim 49, wherein in each iteration of [R″-(p)ₐ-N-(p)_{b}], R″ is independently C₆ alkyl or is absent.

54. The linker compound of claim 49, wherein in each iteration of [R″-(p)ₐ-N-(p)_{b}], R″ is independently 1,4-phenylene, or is absent.

55. The linker compound of any of claims 35-54, wherein in each iteration of [R″-(p)ₐ-N-(p)_{b}], each p is independently a phosphate, phosphorothioate, dithiophosphate, or phosphonate.

56. The linker compound of any of claims 35-55, wherein at least one N is an inverted nucleoside.

57. The linker compound of any of claims 35-55, wherein c is greater than or equal to 2 and at least two Ns are the same nucleoside.

58. The linker compound of claim 57, wherein each N is uridine.

59. The linker compound of claim 57, wherein each N is thymidine.

60. The linker compound of any of claims 35-56, wherein c is greater than or equal to 2 and at least one N is different from another N.

61. The linker compound of any of claims 35-60, wherein B is methanetriyl 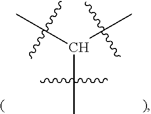 [SMILES: *C(*)*]
ethanetriyl 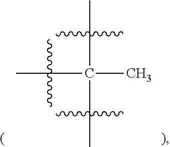 [SMILES: *C(*)(*)C]
propanetriyl 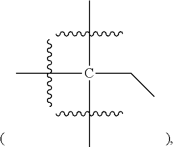 [SMILES: *C(*)(*)CC]
tris(hydroxymethyl)aminomethane, trisubstituted aryl, or substituted ammonia.

62. The linker compound of claim 61, wherein B is methanetriyl 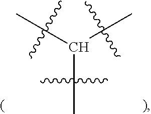 [SMILES: *C(*)*]
ethanetriyl 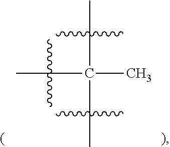 [SMILES: *C(*)(*)C]
propanetriyl 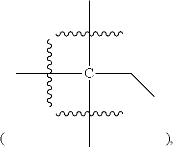 [SMILES: *C(*)(*)CC]
or tris(hydroxymethyl)aminomethane.

63. The linker compound of any of claims 1-17, wherein each nucleotide is independently a naturally-occurring nucleotide, an artificial or non-natural nucleotide analog, or a chemically modified version of any of the foregoing.

64. The linker compound of claim 63, wherein each nucleotide is independently a ribonucleotide or a deoxyribonucleotide.

65. The linker compound of any of claims 18-62, wherein each N is independently a naturally-occurring nucleoside, an artificial or non-natural nucleoside analog, or a chemically modified version of any of the foregoing.

66. The linker compound of claim 65, wherein each N is independently a ribonucleoside or a deoxyribonucleoside.

67. The linker compound of any of claims 1-66, wherein the compound is configured or selected to exhibit higher stability to cleavage by serum nucleases relative to intracellular nucleases.

68. The linker compound of any of claims 1-67, wherein the compound is at least about 75%, at least about 80%, at least about 85%, at least about 90%, at least about 95%, at least about 96%, at least about 97%, at least about 98%, at least about 99%, or about 100% pure.

69. The linker compound of any of claims 1-67, wherein the compound is about 85% to about 95% pure.

70. The linker compound of any of claims 1-67, wherein the compound is greater than or equal to 75% pure; greater than or equal to 85% pure; or greater than or equal to 95% pure.

71. A multimeric oligonucleotide comprising subunits, wherein each of the subunits is independently a single-stranded or double-stranded oligonucleotide, and one or more of the subunits is joined to another subunit via covalent bonds formed by reaction with a compound of any of claims 1-70.

72. The multimeric oligonucleotide of claim 71, wherein each of the subunits is joined to an adjacent subunit via covalent bonds formed by reaction with a compound of any of claims 1-70.

73. The multimeric oligonucleotide of claim 71 or 72, wherein at least two subunits are substantially different.

74. The multimeric oligonucleotide of claim 71 or 72, where all the subunits are substantially the same.

75. The multimeric oligonucleotide of any of claims 71-74, wherein the multimeric oligonucleotide comprises two, three, four, five, or six subunits.

76. The multimeric oligonucleotide of any of claims 71-75, wherein each subunit is 15-30, 17-27, 19-26, or 20-25 nucleotides in length.

77. The multimeric oligonucleotide of any of claims 71-76, wherein one or more subunits are a double-stranded oligonucleotide.

78. The multimeric oligonucleotide of any of claims 71-77, wherein one or more subunits are a single-stranded oligonucleotide.

79. The multimeric oligonucleotide of claim 78, wherein one or more subunits are an antisense oligonucleotide.

80. The multimeric oligonucleotide of claim 77, wherein each subunit is, independently, an siRNA, a saRNA, or a miRNA.

81. The multimeric oligonucleotide of claim 80, wherein each subunit is a double-stranded siRNA.

82. The multimeric oligonucleotide of any of claims 71-81, further comprising a targeting agent.

83. A conjugate comprising a first bioactive compound joined to a second bioactive compound by reaction with a linker compound of any one of claims 1-34.

84. The conjugate of claim 83, wherein each of the first and second bioactive compounds is independently, a peptide, a protein, an oligonucleotide, an organometallic compound, or a small molecule drug.

85. The conjugate of claim 83 or 84, wherein at least one of the bioactive compounds is an oligonucleotide.

86. The conjugate of any of claims 83-85, wherein at least one of the bioactive compounds is an antibody or antibody fragment.

87. The conjugate of claim 86, wherein the antibody is a monoclonal antibody.

88. The conjugate of claim 83, wherein the first bioactive compound is a monoclonal antibody and the second bioactive compound is an oligonucleotide.

89. The conjugate of any of claims 83-88, further comprising a targeting agent.

90. The conjugate of any one of claims 83-89, wherein the conjugate comprises two or more oligonucleotides linked together to form a multimeric oligonucleotide.

91. A multi-conjugate comprising a first, second and third bioactive compound joined together by reaction with a linker compound of any of claims 35-62.

92. The multi-conjugate of claim 91, wherein each of the first, second and third bioactive compounds is independently, a peptide, a protein, an oligonucleotide, an organometallic compound, or a small molecule drug.

93. The multi-conjugate of claim 91 or 92, wherein at least one of the bioactive compounds is an oligonucleotide.

94. The multi-conjugate of claim 91 or 92, wherein two of the bioactive compounds are each independently an oligonucleotide.

95. The multi-conjugate of any of claims 91-94, wherein at least one bioactive compound is an antibody or antibody fragment.

96. The multi-conjugate of claim 95, wherein the antibody is a monoclonal antibody.

97. The multi-conjugate of claim 91, wherein the first bioactive compound is a monoclonal antibody and the second and third bioactive compounds are each independently an oligonucleotide.

98. The multi-conjugate of any of claims 92-97, further comprising a targeting agent.

99. The multi-conjugate of any one of claims 91-98, wherein the multi-conjugate comprises two or more oligonucleotides linked together to form a multimeric oligonucleotide.

100. A method for linking a first compound A to a second compound B comprising the steps of reacting a linker compound according to any of claims 1-34 with A and B, simultaneously or sequentially, under reaction conditions that promote the formation of a first covalent bond between A and the linker compound and a second covalent bond between B and the linker compound.

101. The method of claim 100, wherein A is different from B.

102. The method of claim 101, wherein the functional groups X and X′ on the linker compound are different functional groups.

103. The method of claim 100, wherein A and B are the same.

104. The method of claim 103, wherein the functional groups X and X′ on the linker compound are the same functional groups.

105. The method of any of claims 100-104, wherein A and B are each an oligonucleotide.

106. The method of claim 105, wherein the oligonucleotide is siRNA.

107. The method of claim 100, wherein A is an oligonucleotide or a multimeric oligonucleotide and B is an antibody or antibody fragment.

108. The method of claim 107, wherein the oligonucleotide is siRNA.

109. A method for linking compounds A, B and C together comprising the steps of reacting a linker compound of any of claims 35-62 with each of A, B and C, simultaneously or sequentially, under reaction conditions that promote the formation of a covalent bond between the linker compound and each of A, B and C.

110. The method of claim 109, wherein at least one of A, B and C is different from the other two.

111. The method of claim 110, wherein at least one functional group in the linker compound is a functional group that is different from the other two functional groups.

112. The method of claim 110 or 111, wherein one of A, B and C is an antibody and the other two are oligonucleotides.

113. The method of claim 112, wherein the antibody is a monoclonal antibody and the oligonucleotides are siRNA.

114. The method of claim 109, wherein all three compounds A, B and C are different.

115. The method of claim 114, wherein each functional group in the linker compound is a different functional group.

116. The method of claim 109, wherein all three compounds A, B and C are the same.

117. The method of claim 116, wherein each functional group in the linker compound is the same functional group.

118. A method of treating a disease or condition in a subject comprising the step of administering to the subject an effective amount of a pharmaceutical composition comprising a multimeric oligonucleotide according to any of claims 71-82.

119. A method of treating a disease or condition in a subject comprising the step of administering to the subject an effective amount of a pharmaceutical composition comprising a conjugate according to any of claims 83-89.

120. A method of treating a disease or condition in a subject comprising the step of administering to the subject an effective amount of a pharmaceutical composition comprising a multi-conjugate according to any of claims 91-98.

121. A composition comprising a multimeric oligonucleotide according to any of claims 71-82 and a pharmaceutically acceptable excipient.

122. A composition comprising a conjugate according to any of claims 83-89 and a pharmaceutically acceptable excipient.

123. A composition comprising a multi-conjugate according to any of claims 91-98 and a pharmaceutically acceptable excipient.

124. A composition comprising the multimeric oligonucleotide of any of claims 71-82 for use in the manufacture of a medicament.

125. A composition comprising the conjugate of any of claims 83-89 for use in the manufacture of a medicament.

126. A composition comprising the multi-conjugate of any of claims 91-98 for use in the manufacture of a medicament.

127. A method of modulating activity of a target gene in a cell, the method comprising contacting the cell with a multimeric oligonucleotide according to any of claims 71-82 and maintaining the cell under conditions in which the multimeric oligonucleotide enters the cell and the activity of the target genes is modulated.

128. A method of observing the activity of a bioactive compound in a cell, the method comprising contacting the cell with a conjugate according to any of claims 83-89 and maintaining the cell under conditions in which the conjugate enters the cell and the activity of the bioactive compound is observed.

129. A method of observing the activity of bioactive compound in a cell, the method comprising contacting the cell with a multi-conjugate according to any of claims 91-98 and maintaining the cell under conditions in which the multi-conjugate enters the cell and the activity of the bioactive compound is observed.
